(19) United States
(12) Patent Application Publication
(10) Pub. No.: US 2026/0193604 A1
(43) Pub. Date: Jul. 9, 2026

(54) METHODS OF IDENTIFYING, DIFFERENTIATING BETWEEN, AND/OR SORTING CELLS

(71) Applicant: University of Tasmania, Sandy Bay (AU)

(72) Inventor: Laura Maeve Parsley, Tasmania (AU)

(73) Assignee: University of Tasmania, Sandy Bay (AU)

(21) Appl. No.: 19/132,121

(22) PCT Filed: Nov. 23, 2023
(86) PCT No.: PCT/AU2023/051199

§ 371 Date: May 22, 2025

(30) Foreign Application Priority Data
Nov. 23, 2022 (AU) 2022903552

(51) Int. Cl. C12N 5/071; C12N 5/076; G01N 15/149; G01N 33/50; G01N 33/58; G01N 33/68

(52) U.S. Cl. CPC C12N 5/0612; C12N 5/061; G01N 15/149; G01N 33/5044; G01N 33/582; G01N 33/689; G01N 2405/00

(57) ABSTRACT

The present disclosure relates to methods of identifying, differentiating between and/or sorting sperm cells, or more particularly, X-chromosome bearing sperm (X-CBS) and Y-chromosome bearing sperm (Y-CBS) cells, in a sperm sample, the methods comprising determining a level of a Reactive Oxidant Species and/or a Cellular Change mediated thereby (ROSCC) in each of a plurality of sperm cells in the sperm sample; and based on the determined ROSCC levels, identifying, differentiating between and/or sorting X-CBS and Y-CBS cells in at least a portion of the plurality of sperm cells. Also described are enriched or purified X-CBS and Y-CBS cells, and uses thereof.

### CROSS-REFERENCE TO RELATED APPLICATION

This application is the U.S. National Stage of International Application No. PCT/AU2023/051199, filed Nov. 23, 2023, which was published in English under PCT Article 21(2), which in turn claims the benefit of Australian Provisional Patent Application No. 2022903552, filed on 23 Nov. 2022, the entire contents of which are incorporated herein by reference.

### TECHNICAL FIELD

The disclosure herein relates to methods of identifying, differentiating between, and/or sorting cells. More particularly, the disclosure herein relates to methods of identifying, differentiating between, sorting, separating and/or isolating sex cells and to producing enriched or purified populations of such cells.

### BACKGROUND OF INVENTION

The ability to reliably select offspring sex is desired in many fields of animal production. For example, in the dairy industry, female calves bring financial return, but male calves are a financial burden. In research, gender specific diseases such as testicular or ovarian cancer constrain studies to single sex subjects. In the conservation of endangered species, optimising population sex ratios, e.g., increasing the number of females relative to males, maximises population growth. However, best-practice husbandry protocols employed in animal management result in sex ratios at parity, resulting in animal wastage and inhibiting the success of conservation programs. Despite extensive efforts to develop technology to select offspring sex affordably, reliably, and safely, suitable solutions are lacking.

Offspring sex ratios in research and to a lesser extent agriculture are managed post-partum by killing members of the unwanted sex. Such a strategy raises ethical issues and is also costly in terms of time and money. In dairy cows as an example, gestation averages 9 months. Therefore, farmers lose 9 months of investment in half of their stock. In Australia alone, post-natal sex ratio regulation results in the death of around 400,000 male dairy calves annually which is distressing for the calf and cow and increases the incidence of foetal membrane retention and mastitis.

Despite rigorous regulation of animals used in research, it is difficult to obtain accurate numbers of animal wastage. Data collated from four Australian states in 2018 reported that 5.5 million laboratory mammals were used for scientific and teaching purposes. The number of animals surplus to demand and culled post-partum is not reported, as regulators do not require this information, however, it is possible that the number of animals unnecessarily killed post-partum is in the millions.

Agricultural enterprises circumnavigate equal offspring sex ratios by accessing specialist technology for separating sperm into sub-populations of X- and Y-Chromosome Bearing Sperm (CBS) for assisted reproduction. Current sperm sorting technology relies on the use of variants of the fluorochrome Hoechst, most commonly 33342, which binds to the nucleotide bases adenine and thymine in the minor groove of DNA. Upon nucleotide binding, X- and Y-CBS are identified based on differences in fluorescence intensity, following activation of Hoechst-stained DNA under intensely focused ultraviolet light. X-CBS glow more brightly than Y-CBS, because the total DNA content of X-CBS is larger than Y-CBS, and ranges from 1-7.5% in the mammals studied to date. The larger X chromosome provides more binding sites for Hoechst. The difference in fluorescence intensity measured between the two sperm types is the basis for separation via specialised flow cytometric and microfluidic sorting systems. Purities of X- or Y-CBS ranging from 70-90% correlate to sorting time and likely cost.

Sperm preselection in theory is preferred to post-natal culling, however, Hoechst as a tool in practice poses risks to sperm and offspring. To adequately bind Hoechst dye to DNA, sperm must first be exposed to Hoechst dye for long periods, typically between 45-90 minutes at testicular temperature. After the staining process, sperm are subjected to a lengthy sorting protocol which is damaging to sperm. Long processing times impact survival rates which can be as low as 30% live sperm. Hoechst based sorting is notoriously difficult; thus, sperm cell sorting instrumentation is specialised which can make the equipment prohibitively expensive. Furthermore, the nature of Hoechst-nucleotide interaction causes mutagenicity in some cells, and ultraviolet light is well known to damage cells, and in particular, their DNA. This is further complicated by the lack of efficient DNA repair in sperm cells which as a consequence introduces DNA mutations into the germline of the offspring. While Hoechst technology is the only available option, technical difficulties and the associated risks hinder its widespread use.

Improved methods of identifying and/or sorting cells, such as of sorting sperm into X- and Y-chromosome bearing sperm, that address one or more of the above problems or at least provide a useful alternative, are therefore desirable. In particular, improved fast and accurate methods of identifying and/or sorting sperm that reduce the mutagenicity risk, avoid exposure of sperm to ultraviolet radiation, and/or increase sperm survival are desired.

The invention described herein provides a new method of sorting sperm cells that overcomes the need to use the time consuming and very sensitive sorting processes required to distinguish between total DNA content differences in X- and Y-chromosome bearing sperm cells and that are prone to error as a result of orientation and hydrodynamic effects. The new method supports sperm health and preservation of genetic integrity in sperm DNA by desirably providing a faster, more robust basis on which to differentiate between sperm cells that in preferred embodiments is also non-mutagenic.

A reference herein to a patent document or other matter which is given as prior art is not to be taken as an admission that the document or matter was known or that the information it contains was part of the common general knowledge as at the priority date of any of the claims.

### SUMMARY OF THE INVENTION

Unless the context requires otherwise, where the terms “comprise”, “comprises” and “comprising” are used in the specification (including the claims) they are to be interpreted as specifying the stated features, integers, steps or components, but not precluding the presence of one or more other features, integers, steps or components, or group thereof.

According to a first aspect of the present invention, there is provided a method of sorting sperm cells, comprising: determining a level of a Reactive Oxidant Species and/or a Cellular Change mediated thereby (ROSCC) in each of a plurality of sperm cells; and based on the determined ROSCC levels, sorting at least a portion of the plurality of sperm cells into X-chromosome bearing sperm (X-CBS) and/or Y-chromosome bearing sperm (Y-CBS) cells.

The method may comprise sorting at least a portion of the plurality of sperm cells into X-CBS and Y-CBS cells. In some embodiments, the plurality of sperm cells may comprise a mixture of X-CBS and Y-CBS. The ROSCC may be selected from: a reactive oxidant species selected from: a reactive oxygen species (ROS), a reactive nitrogen species (RNS), and a reactive sulfur species (RSS), or a combination of two or more thereof; and/or a cellular change mediated by a reactive oxidant species selected from: a ROS-induced oxidative change, an RNS-induced oxidative change, and an RSS-induced oxidative change, or a combination of two or more thereof. Determining the level of ROSCC may comprise treating the plurality of sperm cells with a ROSCC detecting agent, in some embodiments, the detecting agent comprises a dye, such as a fluorescing dye. When a fluorescing dye is used as a ROSCC detecting agent, fluorescence may be activated by reaction of the dye with one or more ROSCC in the cells. The ROSCC may be detectable in a component of the sperm cells selected from: a lipid component, such as the cell membrane or mitochondrial membrane; a protein component, such as chromatin or an enzyme; an organelle, such as the mitochondria; an extracellular component, such as ROSCC produced by a cell surface enzyme; the cytoplasm; and DNA, or a combination of any two or more of these components. The ROSCC detecting agent may be selective for a target ROSCC. The detecting agent may detect ROS, RNS and/or RSS in at least the cytoplasm of the sperm cells. The ROSCC detecting agent may detect ROS-, RNS- and/or RSS-induced oxidative damage in a lipid component of the sperm cells. The ROSCC may be a direct product of mitochondrial activity. The ROSCC may be a reactive oxidant species, and the reactive oxidant species may be a ROS selected from superoxide, hydroxyl radical, and hydrogen peroxide. The ROSCC may be a cellular change mediated by a reactive oxidant species, wherein the cellular change is lipid peroxidation.

The level of ROSCC determined in each X-CBS cell may differ by at least 5%, at least 20%, at least 40%, or at least 60% from the level of ROSCC determined in each Y-CBS cell. Alternatively, an average level of ROSCC determined across all cells in the X-CBS may differ by at least 5%, at least 20%, at least 40%, or at least 60% from an average level of ROSCC determined across all cells in the Y-CBS. The level of ROSCC determined in each X-CBS cell may be at least 5%, at least 20%, at least 40%, or at least 60% higher than the level of ROSCC determined in each Y-CBS cell. Alternatively, an average level of ROSCC determined across all cells in the X-CBS may be least 5%, at least 20%, at least 40%, or at least 60% higher than an average level of ROSCC determined across all cells in the Y-CBS.

The plurality of sperm cells may be obtained from the proximal cauda epididymis, the distal cauda epididymis, the vas deferens, or ejaculated semen of a subject. The plurality of sperm cells may be obtained from a mammalian subject. The plurality of sperm cells may be obtained from a non-human mammalian subject.

The sorting may comprise using a flow cytometric sorter or a microfluidic sorting device. The sorting may be based on a visible indicator of ROS level, such as fluorescence or absorbance. The sorting may be based on a physiological function of sperm, such as relative motility. The sorting may utilise magnetic or electrostatic interactions. The sorting may comprise using a flow cytometer, wherein the X-CBS and/or Y-CBS cells are sorted optionally by gating. The determining may be performed in situ in a flow cytometer or microfluidic device prior to sorting. The method may further comprise: providing the sorted X-CBS and/or Y-CBS cells separately in an enriched or purified form. The enriched or purified form may have a minimum purity of at least 90% X-CBS or at least 90% Y-CBS cells. The method may further comprise providing the sorted X-CBS and/or Y-CBS cells separately in a cryopreserved form.

According to a second aspect of the present invention, there is provided an enriched or purified population of X-chromosome bearing sperm (X-CBS) cells sorted according to the method of the first aspect above. The enriched or purified population may have a minimum purity of at least 90% X-CBS cells.

According to a third aspect of the present invention, there is provided an enriched or purified population of Y-chromosome bearing sperm (Y-CBS) cells sorted according to the method of the first aspect above. The enriched or purified population may have a minimum purity of at least 90% Y-CBS cells.

According to a fourth aspect of the present invention, there is provided a method of identifying X-chromosome bearing sperm (X-CBS) or Y-chromosome bearing sperm (Y-CBS) cells in a sperm sample, comprising: determining a level of a Reactive Oxidant Species and/or a Cellular Change mediated thereby (ROSCC) in each of a plurality of sperm cells in a starting sperm sample; and, based on the determined ROSCC levels, identifying at least a portion of the plurality of sperm cells in the starting sperm sample as X-CBS or Y-CBS cells.

The starting sperm sample may comprise a mixture of X-CBS and Y-CBS cells. The method may further comprise isolating at least a portion of the identified X-CBS or Y-CBS cells from the starting sperm sample to produce an enriched sperm sample comprising the at least portion of isolated, identified X-CBS or Y-CBS cells. The enriched sperm sample may comprise at least 90% X-CBS cells, or at least 99% X-CBS cells, or may comprise at least 90% Y-CBS cells, or at least 99% Y-CBS cells. Isolating may comprise using a flow cytometric sorter, optionally with gating, or using a microfluidic sorting device. The method may alternatively further comprise inducing selective motility, infertility or non-viability in either the identified X-CBS or Y-CBS cells in the starting sperm sample to produce a motility, fertility or viability-modified sperm sample. The method may further comprise cryopreserving the enriched sperm sample.

In one embodiment, there is provided a method of isolating X-chromosome bearing sperm (X-CBS) or Y-chromosome bearing sperm (Y-CBS) cells in a sperm sample, comprising: determining a level of a Reactive Oxidant Species and/or a Cellular Change mediated thereby (ROSCC) in each of a plurality of sperm cells in a starting sperm sample; based on the determined ROSCC levels, identifying at least a portion of the plurality of sperm cells in the starting sperm sample as X-CBS or Y-CBS cells; and, isolating at least a portion of the identified X-CBS or Y-CBS cells from the starting sperm sample to produce an enriched sperm sample comprising the at least portion of isolated, identified X-CBS or Y-CBS cells.

In another embodiment, there is provided a method of producing a motility, fertility or viability-modified sperm sample, comprising: determining a level of a Reactive Oxidant Species and/or a Cellular Change mediated thereby (ROSCC) in each of a plurality of sperm cells in a starting sperm sample; based on the determined ROSCC levels, identifying at least a portion of the plurality of sperm cells in the starting sperm sample as X-CBS or Y-CBS cells; and, inducing selective motility, infertility or non-viability in either the identified X-CBS or Y-CBS cells in the starting sperm sample to produce a motility, fertility or viability-modified sperm sample.

According to a fifth aspect of the present invention, there is provided X-CBS or Y-CBS cells identified according to the method of the fourth aspect above.

According to a sixth aspect of the present invention, there is provided use of sperm sorted according to the method of the first aspect above, or an enriched or purified population of X-chromosome bearing sperm (X-CBS) or Y-chromosome bearing sperm (Y-CBS) cells according to the second or third aspects above, or X-CBS or Y-CBS cells identified according to the method of the fourth aspect above, in an assisted reproductive technology.

According to a seventh aspect of the present invention, there is provided a non-human mammalian subject produced from the sperm sorted according to the method of the first aspect above, or an enriched or purified population of X-chromosome bearing sperm (X-CBS) or Y-chromosome bearing sperm (Y-CBS) cells according to the second or third aspects above, or X-CBS or Y-CBS cells identified according to the method of the fourth aspect above.

According to an eighth aspect of the present invention, there is provided a sperm cell sorting system, comprising: a Reactive Oxidant Species and/or a Cellular Change (ROSCC) detecting agent, wherein the Cellular Change is mediated by a Reactive Oxidant Species; and instructions to use the ROSCC detecting agent to determine a level of ROSCC in each of a plurality of sperm cells, and based on the determined ROSCC levels, sort at least a portion of the plurality of sperm cells into X chromosome bearing sperm (X-CBS) and/or Y chromosome bearing sperm (Y-CBS) cells. The sperm cell sorting system may further comprise a sperm cell incubation solution for incubating sperm cells with the ROSCC detecting agent.

According to a ninth aspect of the present invention, there is provided a method of differentiating between X chromosome bearing sperm (X-CBS) and Y chromosome bearing sperm (Y-CBS) cells in a sperm sample, the method comprising: determining a level of a Reactive Oxidant Species and/or a Cellular Change mediated thereby (ROSCC) in each of a plurality of sperm cells in the sperm sample; and based on the determined ROSCC levels, differentiating between X-CBS and Y-CBS in at least a portion of the plurality of sperm cells in the sperm sample.

According to a tenth aspect of the present invention, there is provided a method of differentiating between X chromosome bearing sperm (X-CBS) and Y chromosome bearing sperm (Y-CBS) cells in a sperm sample, the method comprising: determining a level of a Reactive Oxidant Species and/or a Cellular Change mediated thereby (ROSCC) in each of a plurality of sperm cells in the sperm sample; based on the determined ROSCC levels, differentiating between X-CBS and Y-CBS in at least a portion of the plurality of sperm cells in the sperm sample; and isolating at least a portion of the differentiated X-CBS or Y-CBS cells from the sperm sample to produce an enriched or purified sperm sample. The enriched or purified sperm sample may be an enriched or purified population of X-CBS cells. The enriched or purified sperm sample may be an enriched or purified population of Y-CBS cells. The enriched or purified sperm sample may have a minimum purity of at least 90% X-CBS or at least 90% Y-CBS cells. In some embodiments, the isolating uses a flow cytometer or microfluidic sorting device.

In an eleventh aspect of the present invention, there is provided an enriched or purified population of X-CBS cells, or an enriched or purified population of Y-CBS cells, isolated according to the method of the tenth aspect above.

In a twelfth aspect of the present invention, there is provided use of sperm differentiated according to the method of the ninth or tenth aspects above, or of an enriched or purified population of X-CBS or Y-CBS cells according to the eleventh aspect above, in an assisted reproductive technology.

### BRIEF DESCRIPTION OF DRAWINGS

Embodiments of the invention will herein be illustrated by way of example only with reference to the accompanying drawings in which:

FIG. 1 is diagrammatic representation of a testis showing the three locations of the reproductive tract from which sperm were obtained for experiments as described in the Examples herein. The cauda epididymis was halved; section ‘1’ refers to the part of the cauda epididymis adjacent to the corpus epididymis and termed ‘proximal cauda’, section ‘2’ refers to the part of the cauda adjacent to the vas deferens and termed ‘distal epididymis’, and section ‘3’ refers to the vas deferens from the distal cauda epididymis as close to the ejaculatory duct as possible and termed ‘vas deferens’.

FIGS. 2A-2B shows data of sperm sorted with Hoechst 33342 demonstrating the level of separation of X-CBS and Y-CBS that can be achieved in mice of the C57BL/6 strain: a) histogram demonstrating significant overlap in left peak Y-CBS and right peak X-CBS; b) the same data represented as a contour plot.

FIGS. 3A-3B shows data for sperm collected from the proximal cauda epididymis of C57BL/6 mice and sorted into populations of X-CBS and Y-CBS via flow cytometry; a) histogram demonstrating high resolution of left peak Y-CBS and right peak X-CBS identified by differences in mitochondrial superoxide levels via MitoSOX®; b) contour plot of mouse sperm stained and sorted with Hoechst 33342 (vertical axis) and MitoSOX® (horizontal axis.

FIGS. 4A-4B shows data for sperm collected from the distal cauda epididymis of C57BL/6 mice and sorted into populations of X-CBS and Y-CBS via flow cytometry; a) histogram demonstrating high resolution of left peak Y-CBS and right peak X-CBS identified by differences in mitochondrial superoxide levels via MitoSOX®; b) contour plot of mouse sperm stained and sorted with Hoechst 33342 (vertical axis) and MitoSOX® (horizontal axis).

FIGS. 5A-5B shows data for sperm collected from the vas deferens of C57BL/6 mice and sorted into populations of X-CBS and Y-CBS via flow cytometry; a) histogram demonstrating high resolution of left peak Y-CBS and right peak X-CBS identified by differences in mitochondrial superoxide levels via MitoSOX®; b) contour plot of mouse sperm stained and sorted with Hoechst 33342 (vertical axis) and MitoSOX® (horizontal axis).

FIGS. 6A-6C shows data for sperm collected from the vas deferens of C57BL/6 mice and sorted into populations of X-CBS and Y-CBS via flow cytometry; a) histogram of sperm stained with CellROX® Red broad-spectrum cytoplasmic ROS detecting probe; b) histogram of sperm stained with Bodipy C11® lipid peroxidation sensor; and, c) contour plot of sperm stained with Bodipy C11® (vertical axis) and CellROX® Red (horizontal axis), demonstrating the increased resolution of X-CBS and Y-CBS that can be achieved when sorted via lipid peroxidation and cytoplasmic ROS when directly measuring superoxide is not possible.

FIG. 7 shows a bar graph demonstrating the difference in mitochondrial superoxide levels in Y-CBS and X-CBS from C57BL/6 mice as measured by calculating the mean fluorescence intensity (MFI) of 10,000 sperm following staining with MitoSOX® in the proximal and distal cauda epididymis and the vas deferens. On average, the X-CBS emit 7.5 times more fluorescence than Y-CBS in the mouse.

FIGS. 8A-8B shows data for a cryopreserved bull sperm sample sorted into populations of X-CBS and Y-CBS via flow cytometry; a) histogram of sperm stained with MitoSOX® Red; and b) histogram of sperm stained with Hoechst 33342.

FIG. 9 shows data that visualises the large difference in MitoSOX® detected superoxide concentrations between horse X-CBS and Y-CBS cells.

FIG. 10 shows data that visualises the large difference in MitoSOX® detected superoxide concentrations between boar X-CBS and Y-CBS cells.

### DETAILED DESCRIPTION

Described herein is a method of sorting sperm cells, comprising: determining a level of a Reactive Oxidant Species and/or a Cellular Change mediated thereby (ROSCC) in each of a plurality of sperm cells; and, based on the determined ROSCC levels, sorting at least a portion of the plurality of sperm cells into X-chromosome bearing sperm (X-CBS) and/or Y-chromosome bearing sperm (Y-CBS) cells. Also described herein is a method of identifying X-CBS or Y-CBS cells in a sperm sample, comprising: determining a level of a Reactive Oxidant Species and/or a Cellular Change mediated thereby (ROSCC) in each of a plurality of sperm cells in a starting sperm sample; and, based on the determined ROSCC levels, identifying at least a portion of the plurality of sperm cells in the starting sperm sample as X-CBS or Y-CBS cells. This method may further comprise an isolating step to produce a sperm sample enriched in X-CBS or Y-CBS cells, or may comprise a step of inducing selective motility, infertility or non-viability in either the identified X-CBS or Y-CBS cells in the starting sperm sample to produce a motility, fertility or viability-modified sperm sample. The present inventor is the first to discover that X-CBS and Y-CBS differ from each other in their level of ROSCC, and they have advantageously applied this finding to identify, sort and separate X-CBS based on X-CBS cells having a level of ROSCC which is different, such as a higher or lower level of ROSCC, to that of Y-CBS cells. Persons of skill in the art will recognise that identifying X-CBS cells as distinct from Y-CBS cells is equivalent to differentiating between the two different chromosome-bearing cell types. Accordingly, in one embodiment, described herein is a method of sorting sperm cells, comprising: determining a level of a Reactive Oxidant Species and/or a Cellular Change mediated thereby (ROSCC) in each of a plurality of sperm cells; and, based on the determined ROSCC levels, sorting at least a portion of the plurality of sperm cells into X-chromosome bearing sperm (X-CBS) having a level of ROSCC which is different, either at the cell level or on average across all cells, to that of Y-chromosome bearing sperm (Y-CBS). In one embodiment, the level of ROSCC determined in each X-CBS cell differs from the level of ROSCC determined in each Y-CBS cell. In another embodiment, an average level of ROSCC determined across all cells in the X-CBS differs from an average level of ROSCC determined across all cells in the Y-CBS. In one embodiment, the level of ROSCC determined in X-CBS is higher, either at the cell level or on average across all cells, than the level of ROSCC determined in Y-CBS cells. In one embodiment, the term “higher” as used in this context refers to the level of ROSCC determined in X-CBS being at least about 5% greater, either at the cell level or on average across all cells, than the level of ROSCC determined in Y-CBS cells. However, embodiments where the X-CBS has a lower level of ROSCC than the Y-CBS, either at the cell level or on average across all cells, are also contemplated herein. In one embodiment, the term “lower” as used in this context refers to the level of ROSCC determined in X-CBS being at least about 5% less, either at the cell level or on average across all cells, than the level of ROSCC determined in Y-CBS cells.

The level of ROSCC in a given sperm cell is related to the concentration of reactive oxidant species present in that cell, but advantageously, is physiologically detectable in a variety of ways. This opens up the possibility of measuring levels of ROSCC in cells with different ROSCC detecting agents and/or methods to optimise X-CBS and Y-CBS cell identification, separation and/or enrichment. By way of non-limiting example, the level of ROSCC being detected in sperm cells herein may be a level of one or more specific reactive oxidant species, and/or a level of one or more cellular changes mediated by reactive oxidant species caused by reactions of reactive oxidant species with other reactive species and/or cell components. As will be evident in the discussion that follows, the ability to target a range of sperm cell components to measure a level of ROSCC not only offers versatility in terms of which detecting apparatus, detecting agent and sorting apparatus is used, but may also assist in preserving the genetic integrity of sperm DNA. For example, in certain embodiments of the methods described herein, ROSCC detecting agents do not bind to or interfere with DNA in the sperm cells, thereby avoiding potential damage to DNA. In other embodiments, fluorescence of a ROSCC detecting agent used herein is activated without utilising UV light, which avoids problems associated with UV radiation endangering the integrity of, or permanently altering, sperm DNA.

The difference in levels of ROSCC between X-CBS and Y-CBS cells as discovered by the present inventor is surprisingly also conserved in sperm in various stages of sperm maturity occurring from the corpus epididymis to the vas deferens. Determination of level of ROSCC and identification, sorting and enrichment of X-CBS and Y-CBS cells can therefore be performed using the methods herein with sperm cell samples harvested from different locations within the male reproductive system.

Furthermore, unlike previous sperm cell sorting technologies based on the DNA-binding dye Hoechst 33342, the difference in level of ROSCC between X-CBS and Y-CBS cells as discovered by the present inventor is in some embodiments substantially greater than the about 1 to 7.5% difference in DNA payload size between X-CBS and Y-CBS cells. Accordingly, problems caused by the small and variable difference in DNA size, such as the need for high sensitivity and costly sorting instrumentation (including specialist lasers), may be advantageously overcome in certain embodiments of the methods described herein.

The magnitude of the difference in level of ROSCC between X-CBS and Y-CBS may additionally make sperm cells easier to identify and detect compared to using Hoechst 33342, resulting in shorter sorting times and less potential damage to sperm and therefore potentially increased fertilisation rates and healthier offspring. By way of explanation, sperm are time-critical cells that lose effectiveness the longer they remain unused. However, to ensure small differences in DNA size are sufficiently identified for sorting, sperm must be exposed to Hoechst variants for a period of up to 1.5 h in some species. Further, the small differences in DNA content between X-CBS and Y-CBS (1-7.5% in the mammals studied to date) translate to overlapping peaks in fluorescence, which in turn lead to slow sorting times of up to 4 hrs. The extended staining and sorting times associated with methods based on this DNA-binding dye can thus be detrimental to the recovery rates of the sorted sperm. By contrast, in certain embodiments of the methods described herein, staining of sperm to ROSCC is completed in 10 min, and in other embodiments, in 30 min. The magnitude of the difference between the level of ROSCC in X-CBS and Y-CBS cells is substantially easier to detect than difference in DNA size, which in turn allows for shorter identification/sorting times compared to using DNA-binding dyes like Hoechst 33324 and other variants.

Furthermore, unlike DNA-based dye detection methods in which fluorescence events may be observed for damaged or dead sperm cells, in certain embodiments, the methods herein advantageously selectively produce a signal, such as dye response, only in living cells with functional metabolisms. Such embodiments avoid the need for signal interference corrections caused by signal detection from dead sperm in samples, particularly in the case of cryopreserved sperm samples containing dead and/or damaged sperm resulting from the freezing process.

Although agents for detecting certain reactive oxygen species in cells are known, and methods have been reported that utilise these dyes for the purpose of assessing sperm health generally, the link between ROSCC and X- or Y-chromosome carriage in sperm cells has never before been appreciated. Furthermore, although ligand activation of Toll-like receptors 7/8 (TLR7/8) was used in one study to suppress mobility of X-chromosome bearing sperm and thereby allow separation of X- from Y-chromosome bearing sperm (Umehara et al., PLoS Biol. 2019 Aug. 13; 17(8)), there is no relationship between TLR7/8 and reactive oxygen species production in sperm. Accordingly, the presently described methods represent a significant advance in the art.

The methods described herein are suitable for sorting a plurality of sperm cells. The term “plurality” encompasses two or more sperm cells. Accordingly, in some embodiments, the methods described herein are suitable for use in single cell sorting methods. In other embodiments, the methods described herein are suitable for use in multiple cell sorting methods. When employing automated cell sorting apparatus, the term “plurality” may refer to in excess of 10², or 10³, or 10⁴, or 10⁵, or 10⁶, or 10⁷ sperm cells, or in excess of 10², or 10³, or 10⁴, or 10⁵, or 10⁶, or 10⁷ sperm cells/mL. The methods herein are suitable for sorting at least a portion of a plurality of sperm cells into X-chromosome bearing sperm (X-CBS) and/or Y-chromosome bearing sperm (Y-CBS). The plurality of sperm cells herein, prior to sorting, desirably comprises a mixture of X-CBS and Y-CBS cells. This advantageously allows the relative level of ROSCC in X-CBS vs. Y-CBS cells to be determined and therefore for the X-CBS cells (or a portion thereof) to be identified and separated from the Y-CBS cells (or a portion thereof). In some embodiments, the plurality of sperm cells prior to sorting according to the methods described herein comprises about 40-60% X-CBS cells and 60-40% Y-CBS cells, such as approximately 50% X-CBS cells and 50% Y-CBS cells, or in other embodiments comprises any other naturally occurring X-CBS:Y-CBS cell ratio. In some embodiments, the plurality of sperm cells may be pre-sorted or pre-enriched prior to sorting according the methods described herein, such that the sperm cells have a non-naturally occurring X-CBS:Y-CBS cell ratio. Desirably, if used, pre-sorted or pre-enriched sperm cells comprise a mixture of X-CBS and Y-CBS cells. In some embodiments, pre-sorting or pre-enrichment may be performed according to a method described herein, or in other embodiments may be performed using another method, such as a Hoechst-based method.

Use of the term “at least a portion” is used herein to clarify that not every cell in the plurality of sperm cells needs to be identified or sorted as an X-CBS or a Y-CBS cell in the described methods. By way of further explanation, some sperm cells may not be identified or sorted into either X-CBS or Y-CBS cells because they carry variants such as chromosomal diploidy, or are damaged or fragmented during sorting, are inadequately detected, or have ROSCC levels that are insufficiently determinative of an X-CBS or Y-CBS cell using a particular technique/detecting agent, etc.

The sperm cells for use in the methods herein may be obtained from any suitable source. In one embodiment, the sperm cells are obtained from within the reproductive system of a subject, such as from the epididymis, for example corpus epididymis, of a subject, or from the vas deferens. In one embodiment, the sperm cells are obtained from the proximal cauda epididymis of a subject. In another embodiment, the sperm cells are obtained from the distal cauda epididymis of a subject. In a further embodiment, the sperm cells are obtained from the vas deferens of a subject. In yet a further embodiment, the sperm cells are obtained from ejaculated semen from a subject. In another embodiment, the sperm cells are obtained from the proximal cauda epididymis, the distal cauda epididymis, the vas deferens, or from ejaculated semen from a subject.

Persons of skill in the art will be familiar with methods to isolate sperm cells in a seminal fluid from the seminal plasma in order to provide a sample of sperm cells suitable for sorting using the methods herein. Methods to isolate sperm cells from seminal plasma generally comprise combining the seminal fluid containing sperm cells with a suitable aqueous solution, such as a buffer, including phosphate buffered saline (PBS) at pH ~7.4, semen extender, human tubule fluid, cell culture media, media for assisted reproduction, or the like, centrifuging the aqueous sperm mixture, discarding the supernatant, and resuspending the sperm cells in a suitable aqueous solution. In some embodiments, the resuspended sperm cells may be optionally incubated with a detecting agent and then washed and resuspended prior to detecting and sorting. In some embodiments, the sperm sample used in the methods herein is one in which sperm cells have been isolated from seminal plasma prior to determining their level of ROSCC. Such embodiments advantageously reduce interference during measurement by seminal plasma components, assist in formulating clean, enriched sperm samples for use, and/or prolong functionality of sperm. In other embodiments, the sperm sample may be separated without first pre-isolating the sperm cells from the seminal fluid. In some embodiments, use of a ROSCC indicator or dye selective for specific endogenous ROSCC may avoid effects of any seminal fluid components on the measured ROSCC levels of the sperm cells in the sample.

The sperm cells for use in the methods described herein may be obtained from any suitable subject. In one embodiment, the sperm cells are obtained from a mammalian subject. In one embodiment, the mammalian subject is a domesticated animal, such as dog, cat, cattle, horse, sheep, donkey, or camel. In one embodiment, the mammalian subject is cattle. In one embodiment the mammalian subject is a livestock animal, such as a horse, sheep, cattle, or goat. In one embodiment the mammalian subject is a research animal, such as a mouse, rat, non-human primate, guinea pig, rabbit or hamster. In one embodiment, the mammalian subject is a human. In one embodiment, the mammalian subject is an endangered or threatened mammalian species, such as a rhinoceros, tiger, leopard, gorilla, elephant, orangutan, panda, etc. In one embodiment, the sperm cells are obtained from a non-human mammalian subject. In another embodiment, the sperm cells are obtained from a non-mammalian heterogametic male vertebrate.

The term “Reactive Oxidant Species and/or a Cellular Change mediated thereby” or “ROSCC” as used herein refers to any reactive oxidant species or cellular change mediated by reactive oxidant species, including species that are either reactive oxidants themselves or species that are produced by reaction(s) of a reactive oxidant with another chemical species, such as with another reactive oxidant and/or with a cell component. As used herein, the term “reactive” means that the ROSCC readily undergoes chemical reactions with at least one other species, such as species present in the cell environment, and may refer to radical species containing an unpaired electron, such as the hydroxyl radical (·OH), or to non-radical species, such as hydrogen peroxide (H₂O₂). As used herein, the term “oxidant” means that the ROSCC reacts with (oxidises) other species, gaining one or more electrons in the process.

Species that are produced by reaction(s) of a reactive oxidant with a cell component may be referred to herein as a “cellular change mediated by a reactive oxidant species”. For the avoidance of doubt, the cellular change may be a product formed in a single reaction between a cell component and a reactive oxidant, or may form after a chain of reactions in one or more cell components triggered by a reactive oxidant. Cell components susceptible to reactions with reactive oxidant species include lipids, proteins, carbohydrates, and nucleic acids. It will be appreciated that some cellular changes mediated by a reactive oxidant species may be products of oxidative attack, and some cell components may be transformed into reactive oxidant species during this process. Accordingly, in some embodiments, there is overlap in what is considered a reactive oxidant species and what is considered a cellular change mediated by a reactive oxidant species. The term “ROSCC” is thus used herein for convenience to emphasise that the present invention is not limited to sorting based on detection of reactive oxidant species produced directly by metabolic processes, primarily being ·OH, ·O₂⁻, and NO·, but readily includes sorting based on detection of reactive oxidant species formed when ·OH, ·O₂⁻, and/or NO· react with other radicals, ions, and/or molecules in the cell to cause cellular change.

The nature of the ROSCC whose level is determined in the methods herein is not intended to be particularly limited, provided that it is a ROSCC whose level is determinable in sperm cells. In one embodiment, the ROSCC whose level is determined in the methods herein is advantageously one whose level differs significantly between X-CBS and Y-CBS cells. In another embodiment, the ROSCC whose level is determined in the methods herein is advantageously one whose level differs enough to differentiate a subpopulation of X-CBS cells in a sperm cell sample, to differentiate a subpopulation of Y-CBS cells in a sperm cell sample, or to differentiate a subpopulation of X-CBS cells in a sperm cell sample from a subpopulation of Y-CBS cells in a sample, optionally wherein the subpopulations of X-CBS and/or Y-CBS cells form only a portion of the plurality of sperm cells initially designated for sorting.

As used herein, the “level” of ROSCC is intended to cover any means of quantifying an amount or concentration of ROSCC. The level of ROSCC as determined in each sperm cell will generally not be the total concentration of ROSCC (either any single species or a combination thereof) in the cell. Instead, the level of ROSCC as determined in each sperm cell in some embodiments is indicative of a total ROSCC level (either any single species or a combination thereof) in the cell, in some embodiments proportionally indicative of a total ROSCC level (either any single species or a combination thereof) in the cell. In embodiments where a detecting agent is used, this may refer to the detecting agent being used in a concentration substantially lower than the concentration of ROSCC (either any single species or a combination thereof) in the cells, such as at a concentration of detecting agent of less than 1 μM, or less than 1.5 μM, or less than 2.0 μM, or less than 5 μM, or less than 10 μM, or less than 50 μM, or of from 0.01 to 50 μM, or of from 0.1 to 25 μM, or of from 0.5 to 5 μM. Advantageously, such embodiments avoid altering, or substantially altering, the total level of ROSCC in the cell when determining the level of ROSCC for the purposes of sorting the cells into X-CBS and/or Y-CBS cells. For completeness, there are detecting agents known to have catalytic neutralising activity towards one or more ROSCC, which may therefore be capable of altering the ROSCC levels inside cells, and which may be suitable for use in certain embodiments of the present invention.

It will be understood that each sperm cell in the plurality of sperm cells in a given sample desirably has its ROSCC level determined using the same methodology in order to compare ROSCC levels between X-CBS and Y-CBS cells in the given sample and therefore enable sorting/separation into X-CBS and/or Y-CBS cells. In some embodiments, the level of ROSCC is a concentration. In some embodiments, the level of ROSCC is a detectable level of ROSCC, such as the level detectable by a specific technology, and if applicable, detecting agent. In one embodiment, the level of ROSCC is a level of fluorescence intensity, such as a detectable fluorescence intensity. In another embodiment, the level of ROSCC is a level of optical density or absorbance, such as a detectable level of optical density or absorbance. In some embodiments, the level of ROSCC is a relative level of ROSCC.

In one embodiment, the ROSCC is a reactive oxidant species. In one embodiment, the ROSCC is a cellular change mediated by a reactive oxidant species. In one embodiment, the ROSCC is an endogenous reactive oxidant species and/or cellular change mediated by an endogenous reactive oxidant species. In another embodiment, the ROSCC encompasses at least reactive oxygen species (ROS), reactive nitrogen species (RNS), reactive sulfur species (RSS), ROS-mediated cellular change, RNS-mediated cellular change, and/or RSS-mediated cellular change. In one embodiment, the level of ROSCC is a level of reactive oxygen species (ROS), reactive nitrogen species (RNS) or reactive sulfur species (RSS), or a combination of any two or more of these. Reactive oxygen species (ROS) include the superoxide anion radical (·O₂⁻), hydroxyl radical (·OH), hydroperoxyl radical (HO₂·), singlet oxygen (¹O₂), ozone (O₃), and hydrogen peroxide (H₂O₂). Reactive nitrogen species (RNS) include nitric oxide radical (NO·), peroxynitrite (ONOO⁻) and nitrogen dioxide (NO₂). Reactive sulfur species (RSS) include hydrogen sulfide (H₂S), small-molecular-weight thiols (RSH), hydrogen persulfides/polysulfides (H₂Sₙ; n≥2), small-molecular-weight thiol persulfides (RSSH), protein persulfides (PS-SH), various polysulfides (RSS₍ₙ₎H, RSS₍ₙ₎R, and H₂Sₙ; n>1), sulfenic acids (RSOH), nitrosothiols (RSNO), and various sulfide bridge forms (PS—SR, RS—S—SR, and RS—Sₙ—SH). In another embodiment, the level of ROSCC is a level of ROS-mediated cellular change, RNS-mediated cellular change, and RSS-mediated cellular change, or a combination of any two or more of these. In one embodiment, the cellular change mediated by a reactive oxidant species is selected from a ROS-induced oxidative change, an RNS-induced oxidative change, and an RSS-induced oxidative change, or a combination of two or more thereof.

In one embodiment, the reactive oxidant species whose level is determined in the methods herein is any suitable reactive oxidant species. In one embodiment, the level of ROSCC is the level of a single reactive oxidant species. In another embodiment, the level of ROSCC is a level of a mixture of two or more reactive oxidant species. In one embodiment, the reactive oxidant species is a radical species. In such embodiments, the radical species may be a superoxide anion radical (·O₂⁻), a hydroxyl radical (·OH), a nitric oxide radical (NO·), or a hydroperoxyl radical (HO₂·). The level of radical species in a sperm cell may be determined using a technique, and as applicable, detecting agent, as outlined in Table 1 below. The radicals may be detected in any suitable component of a sperm cell, such as in a lipid component, a protein component, an organelle, an extracellular component, the cytoplasm, or in DNA. In one embodiment, the radical species is a primary radical species, being a direct product of cellular metabolic or respiratory activity. In such embodiments, the primary radical may be as a superoxide radical ion (·O²⁻), a hydroxyl radical (·OH), or a nitric oxide radical (NO·). In another embodiment, the reactive oxidant species is a non-radical species. In such embodiment, the non-radical species may be singlet oxygen (¹O₂), ozone (O₃), hydrogen peroxide (H₂O₂), hypochlorous acid (HOCl), or peroxynitrite (ONOO⁻). The level of non-radical species in a sperm cell may be determined using a technique, and if applicable, detecting agent, as outlined in Table 1 below. The radicals may be detected in any suitable component of a sperm cell, such as in a lipid component, a protein component, an organelle, an extracellular component, the cytoplasm, or in DNA. In one embodiment, the reactive oxidant species is a reaction product of a primary radical with another radical, ion or molecule. In such embodiments, the reaction product may be a hydroperoxyl radical (HO₂·), peroxynitrite (ONOO⁻), hypochlorous acid (HOCl), or hydrogen peroxide (H₂O₂). In one embodiment, the ROSCC is a reaction product of two ROS, RNS or RSS, such as peroxynitrite (ONOO⁻), which is a reaction product of the ROS ·O²⁻ and the RNS NO·. In one embodiment, the ROSCC is superoxide, hydroxyl radical, and/or hydrogen peroxide.

TABLE 1
Table of selected techniques and detecting agents suitable for use in
determining levels of ROSCC according to the methods described herein

| | | | Cell component where |
|---|---|---|---|
| Technique | Detecting agent | Species detected | species is detected |
| Fluorescence | N-acetyl-3,7- | H₂O₂ | Extracellular |
| (may be used in | dihydroxyphenoxazine | | |
| conjunction with | (Amplex Red) | | |
| flow cytometry, | mitoSOX ® Red | •O₂⁻ | Organelle (mitochondria) |
| live-cell imaging, | (Invitrogen) | | |
| confocal imaging, | MitoRed (Sigma | •O₂⁻ | Organelle (mitochondria) |
| fluorescence | Aldrich) | | |
| microscopy, etc) | CellROX ® green | •O₂⁻, •OH | DNA (nucleus) |
| | (Invitrogen) | | |
| | Biotinylated glutathione | glutathiolation | Protein (glutathiolation) |
| | ethyl ester (BioGEE) | | |
| | Dihydrorhodamine | ONOO⁻ | Cytoplasm/chromatin |
| | (DHR) | | |
| | Dihydroethidium (DHE) | •O₂⁻ | Cytoplasm/chromatin |
| | Dichlorodihydrofluorescein | H₂O₂, L-OO•, | Cytoplasm |
| | diacetate (DCFH-DA) | L-OOH•, ONOO⁻ | |
| | CellROX ® deep red | •O₂⁻, •OH | Cytoplasm |
| | (Invitrogen) | | |
| | CellROX ® orange | •O₂⁻, •OH, NO•, | Cytoplasm |
| | (Invitrogen) | H₂O₂, ONOO⁻ | |
| | Singlet Oxygen Sensor | ¹O₂ | Cytoplasm |
| | Green | | |
| | Trans-1-(2′- | ¹O₂ | Cytoplasm |
| | Methoxyvinyl)pyrene | | |
| | CM-H2DCFDA | •O₂⁻, •OH, HO₂•, | Cytoplasm |
| | (Invitrogen) | ¹O₂, O₃, H₂O₂, NO• | |
| | Aminophenyl | H₂O₂, NO•, HOCl | Plasma membrane, |
| | fluorescein (APF) | | Cytoplasm |
| | Hydroxyphenyl | •OH, ONOO⁻, | Cytoplasm |
| | fluorescein (HPF) | HOCl, HOBr | |
| | Parinaric acid | L-OO•, L-OOH• | Lipid (lipid peroxidation) |
| | Diphenyl-1- | L-OOH• | Lipid (lipid peroxidation) |
| | pyrenylphosphine | | |
| | (N-(aminooxyacetyl)-N′- | 4-hydroxy-2- | Lipid (lipid peroxidation) |
| | (D-Biotinoyl) hydrazine | nonenal (HNE) | |
| | BODIPY ® 665/676 | L-OO•, L-OOH• | Lipid (lipid peroxidation) |
| | (Invtirogen) | | |
| | BODIPY ®-C11 | L-OOH | Lipid (lipid peroxidation) |
| | (581/591) (Invitrogen) | | |
| | Luminol | H₂O₂, NO•, HOCl | Plasma membrane, |
| | | | cytoplasm |
| Positron emission | [¹⁸F]-radiolabelled | •O₂⁻ | Varies depending on |
| tomography (PET) | agent such as DHE, a | | DHE/analogue selected |
| | DHE analogue | | |
| Electron | — | NO• | Varies depending on |
| paramagnetic | | | molecule¹ |
| resonance EPR/ | 5,5-dimethyl-1- | •O₂⁻ | Endosomes, Lysosomes |
| Electron spin | pyrroline-N-oxide | | |
| resonance (ESR)² | N-tert-butyl-α- | •O₂⁻ | Endosomes, Lysosomes |
| | phenylnitrone | | |
| | 2,2,6,6-tetramethyl- | •O₂⁻ | Endosomes, Lysosomes |
| | piperidine-1-oxyl radical | | |
| Spectroscopy | 2,4-dinitrophenyl- | R₂C═O, RC═O | Protein (carbonylation) |
| | hydrazine (DNPH) | | |

¹Maia L B, Moura J J. Methods Mol Biol. 2016; 1424: 81-102; Berliner L J, Fujii H. Antioxid Redox Signal. 2004; 6(3): 649-56.
²Abbas K, et al. Methods. 2016 Oct. 15; 109: 31-43; for localised detection of radicals, can becombined with antibodies, see: Mason R P. Redox Biol. 2016 August; 8: 422-9.

In another embodiment, the ROSCC is a cellular change mediated by a reactive oxidant species. The cellular change mediated by a reactive oxidant species may be any suitable cellular change. By “cellular change”, it is meant any change, but generally a chemical change, that occurs within or to a cell or any cellular component thereof that is mediated by a reactive oxidant species. By “mediated”, it is meant that the reactive oxidant species initiates the cellular change, optionally via formation of a secondary or higher-order reactive by-product, or otherwise participates in the cellular change pathway in a manner that contributes to bringing about the cellular change.

In one embodiment, the cellular change is oxidative and/or nitrosative damage. In one embodiment, the ROSCC is a cellular change mediated by a reactive oxidant species, and the cellular change is lipid peroxidation, protein (amino acid) oxidation, or DNA oxidation. In one embodiment, the ROSCC is a cellular change mediated by a reactive oxidant species and the cellular change is lipid peroxidation. Lipid peroxidation refers to the process whereby reactive species, especially ·OH and HO₂· radicals, attack lipids (L) containing carbon-carbon double bonds such that oxygen is inserted into the lipid chains and result in formation of lipid peroxyl radicals (LOO·) and hydroperoxides (L-OOH). Levels of lipid peroxy radicals and hydroperoxides can be determined in sperm using any suitable method known to those of skill in the art. One or more suitable techniques or agents is shown above in Table 1.

In another embodiment, the ROSCC is a cellular change mediated by a reactive oxidant species, and the cellular change is DNA oxidation. DNA oxidation can take several forms, but may occur as hydrogen abstraction from the deoxyribose sugar backbone and/or bases, oxidation of bases, and strand excision, often involving ·OH and O₂. Levels of DNA oxidation can be determined in sperm using any suitable method known to those of skill in the art. One or more suitable techniques or agents is shown above in Table 1.

In a further embodiment, the ROSCC is a cellular change mediated by a reactive oxidant species, and the cellular change is protein oxidation. In one embodiment, the protein oxidation comprises oxidation of one or more amino acids comprising a protein either in their side chains or backbone. In one embodiment, the protein oxidation is amino acid carbonylation. Protein carbonylation occurs when reactive oxidant species attack the amino acid side chains of proline, arginine, lysine, and threonine in presence of transition metals to form reactive ketones or aldehydes, which can then react further with other amino acid residues. In another embodiment, the protein oxidation is thiol oxidation. Thiol oxidation occurs when thiol (—SH) side chains on amino acid residues such as cysteine and methionine are oxidised to form disulfide bridges (—SS—). Proteins in sperm containing thiol groups and therefore susceptible to thiol oxidation include enzymes, antioxidant molecules, and structural proteins in the axoneme of sperm. In one embodiment, the protein oxidation is amino acid nitration, such as nitration of tyrosine. Other forms of protein oxidation suitable for use in the methods of the present invention, such as tyrosine nitration, S-nitrosylation, S-glutathionylation, 4-HNE protein adducts, etc. will be known to those in the art. Levels of protein oxidation can be determined in sperm using any suitable method known to those of skill in the art. One or more suitable techniques or agents is shown above in Table 1.

In one embodiment, the ROSCC is a combination of two or more specific reactive oxidant species, such as described above, or is a combination of two of more types of cellular change mediated by a reactive oxidant species, such as described above, or is a combination of one or more specific reactive oxidant species and one or more types of cellular change. In embodiments where the ROSCC comprises a combination of species and/or cellular changes, the level of ROSCC may be the sum of all species/cellular changes measured. In some embodiments, the specific ROSCC determined in the methods herein will be driven by the choice of apparatus, and if applicable, detecting agent. By way of example only, if a fluorescent dye such as MitoSOX® (Invitrogen) is used in a flow cytometry system, the level of ROSCC may be a level of mitochondrial superoxide quantified by fluorescence intensity.

The source of ROSCC whose level is determined in sperm cells according to the methods described herein is not particularly limited as described above. The ROSCC may be endogenous ROSCC produced by cellular processes such as respiration and metabolism, where it may arise from activity of the mitochondria, an enzyme such as nicotinamide adenine dinucleotide phosphate (NADPH) oxidase (NOX), xanthine oxidase (XO), nitric oxide synthase (NOS) or cytochrome P450, or the mitochondrial electron transport chain (ETC) within sperm cells.

In one embodiment, the ROSCC is detectable in a component of the sperm cells selected from a lipid component, a protein component, an organelle, an extracellular component, the mitochondria, the cytoplasm, and DNA, or a combination of any two or more of these components. In one embodiment, the ROSCC is selectively detectable in component of the sperm cells selected from a lipid component, a protein component, an organelle, an extracellular component, the mitochondria, the cytoplasm, and DNA. In one embodiment, the ROSCC is detectable in two or more components of the sperm cells selected from a lipid component, a protein component, an organelle, an extracellular component, the mitochondria, the cytoplasm, and DNA.

In one embodiment, the ROSCC is a direct or indirect product of mitochondrial activity in the sperm cells. In some embodiments, a detecting agent is used that is selective for ROSCC produced by the mitochondria of the sperm cells. Non-limiting examples of suitable measurement techniques and, if appropriate, detecting agents, able to determine ROSCC produced by mitochondria are outlined in Table 1.

In one embodiment, the level of ROSCC corresponds to a level of ROSCC detectable within an organelle of the sperm cells. In one embodiment, the level of ROSCC corresponds to a level of ROSCC detectable within the mitochondria of the sperm cells. As mitochondria are located in the sperm midpiece, embodiments that determine levels of reactive oxidant species in or produced by the mitochondria may be less susceptible to sperm orientation effects caused by sperm heads obstructing each other, and may therefore advantageously allow easier and/or more accurate sorting than a level of ROSCC detectable in the sperm heads. In some embodiments, a detecting agent is used that is selective for ROSCC within the mitochondria of the sperm cells. Non-limiting examples of suitable measurement techniques and, if appropriate, detecting agents, able to determine ROSCC levels within organelles, such as mitochondria, are outlined in Table 1, but other suitable techniques and/or agents may be known to those of skill in the art. In one embodiment, the level of ROSCC corresponds to a level of ROSCC detectable within the midpiece of the sperm.

In another embodiment, the level of ROSCC corresponds to a level of ROSCC detectable within the cytoplasm of the sperm cells. In some embodiments, a detecting agent is used that is selective for ROSCC within the cytoplasm of the sperm cells. Non-limiting examples of suitable measurement techniques and, if appropriate, detecting agents, able to determine ROSCC levels within the cytoplasm of sperm cells, are outlined in Table 1, but other suitable techniques and/or agents may be known to those of skill in the art.

In another embodiment, the level of ROSCC corresponds to a level of ROSCC detectable within a lipid component of the sperm cells. In some embodiments, the lipid component comprises cell and mitochondrial membranes. In some embodiments, a detecting agent is used that is selective for ROSCC within the lipid component of the sperm cells, such as selective for oxidative changes within the cell membrane and/or the mitochondrial membrane. Non-limiting examples of suitable measurement techniques and, if appropriate, detecting agents, able to determine ROSCC levels within a lipid component of sperm cells, are outlined in Table 1, but other suitable techniques and/or agents may be known to those of skill in the art.

In a further embodiment, the level of ROSCC corresponds to a level of ROSCC detectable within a protein component of the sperm cells. In some embodiments, the protein component comprises enzymes. In some embodiments, the protein component may be the protein component of chromatin. In some embodiments, a detecting agent is used that is selective for ROSCC within a protein component of the sperm cells, such a selective for protein oxidation, including protein carbonylation. Non-limiting examples of suitable measurement techniques and, if appropriate, detecting agents, able to determine ROSCC levels within a protein component of sperm cells, are outlined in Table 1, but other suitable techniques and/or agents may be known to those of skill in the art.

In yet a further embodiment, the level of ROSCC corresponds to a level of ROSCC detectable in an extracellular component of the sperm cells. The extracellular component may refer to the immediate extracellular space around a sperm cell. In some embodiments, extracellular ROSCC result from activity of a protein component of the sperm cells embedded in and outward facing from the cell membrane. In one embodiment, the protein component of the sperm cells embedded in the cell membrane is a cell surface enzyme, such as NOX. Accordingly, in such embodiments, ROSCC may be detected in the extracellular rather than intracellular space. In some embodiments, a detecting agent is used that is selective for ROSCC in the extracellular component of the sperm cells. Non-limiting examples of suitable measurement techniques and, if appropriate, detecting agents, able to determine ROSCC levels in an extracellular component of sperm cells, are outlined in Table 1, and include WST-1 dye containing a lipid linker to anchor into the cell membrane, but other suitable techniques and/or agents may be known to those of skill in the art.

In a further embodiment, the level of ROSCC corresponds to a level of ROSCC detectable within DNA of the sperm cells. In some embodiments, a detecting agent is used that is selective for ROSCC within DNA of the sperm cells, such a selective for hydrogen abstraction or oxidation of bases and/or deoxyribose sugar moieties. Non-limiting examples of suitable measurement techniques and, if appropriate, detecting agents, able to determine ROSCC levels within the DNA of sperm cells, are outlined in Table 1, but other suitable techniques and/or agents may be known to those of skill in the art.

In one embodiment, determining the level of ROSCC comprises treating the plurality of sperm cells with a detecting agent that detects ROS, RNS and/or RSS in at least the cytoplasm of the sperm cells. In another embodiment, determining the level of ROSCC according to the methods herein comprises treating the plurality of sperm cells with a ROSCC detecting agent that detects ROS-, RNS- and/or RSS-mediated cellular changes in a lipid component of the sperm cells.

Persons of skill in the art will readily appreciate that a ROSCC and corresponding measuring technique including applicable detecting agent may be identified as suitable for use in identifying sperm cells as, and/or sorting sperm cells into, X-CBS and Y-CBS cells, or in producing samples enriched in X-CBS or Y-CBS cells, by confirming that the determined levels of ROSCC differ detectably between X-CBS and Y-CBS cells or at least a portion thereof. In some embodiments, a ROSCC and corresponding measuring technique including applicable detecting agent may be selected such that there is no overlap in the determined ROSCC levels in X-CBS and Y-CBS cells in a sample. In another embodiment, a ROSCC and corresponding measuring technique including applicable detecting agent may be identified as suitable for use in identifying sperm cells as, and/or sorting sperm cells into, X-CBS and Y-CBS cells, or in producing samples enriched in X-CBS or Y-CBS cells, by confirming that the determined levels of ROSCC differ enough to differentiate a subpopulation of X-CBS cells from a subpopulation of Y-CBS cells, generally wherein the subpopulations of X-CBS and Y-CBS cells form only a portion of the plurality of sperm cells initially designated for identification, sorting or enriching. In such embodiments, a ROSCC and corresponding measuring technique including applicable detecting agent may be selected such that there is some overlap in the levels of ROSCC between some X-CBS and Y-CBS cells in a sample, but the overlap affects a sufficiently small portion of the sample as to permit identification, separation and/or collection of X-CBS and/or Y-CBS cells separately, in non-overlapping regions, through processes such as gating, or permits inducing of selective motility, infertility or non-viability in either identified X-CBS or Y-CBS cells in the sperm sample.

Relatedly, it will be understood that a combination of measurement techniques and, as appropriate, detecting agents, may be used in the methods described herein to identify and/or sort sperm. In one embodiment, two or more detecting agents are used simultaneously or sequentially to determine ROSCC levels and therefore identify and/or sort sperm in the methods herein. In another embodiment, two or more techniques and optionally one or more detecting agents are used sequentially to determine ROSCC levels and therefore identify and/or sort sperm in the methods herein.

As will be evident from the range of techniques listed in Table 1, the means of determining the level of ROSCC in sperm cells is not particularly limited in the methods herein. In some embodiments, the level of ROSCC is measured by spectroscopic means. Spectroscopic means may include fluorescence spectrophotometry and photometry and related technologies, optionally combined with microscopy or other imaging technologies such as tomography. In some embodiments, the spectroscopic means are combined with techniques to assist in viewing, processing and/or aligning cells, such as image cytometry, flow cytometry or microfluidics. Other means of measuring the level of ROSCC will be known to those of skill in the art, including but not limited to measuring a physiological indicator of ROSCC level, such as sperm motility.

In some embodiments, the methods herein comprise determining a level of ROSCC in each of a plurality of sperm cells, wherein the step of determining comprises treating the plurality of sperm cells with a ROSCC detecting agent. The ROSCC detecting agent may detect reactive oxidant species, or may detect cellular changes mediated by reactive oxidant species, or may detect a combination of the two. In one embodiment, the detecting agent comprises a dye. In some embodiments, the detecting agent, optionally a dye, detects the ROSCC directly, such as interacts with the ROSCC to produce a detectable (such as visible) response indicative of the presence and quantity of one or more ROSCC. In some embodiments, light, radiation or other stimulus may be required to activate the detectable response. In one embodiment, the detecting agent is activated, such as activated to produce a detectable signal, by light. In one embodiment, the detecting agent used herein is activated to produce a detectable signal by visible light, such as having a wavelength in the range of from 400 nm to 700 nm. Examples of suitable detecting agents activated by visible light include fluorescing dyes MitoSOX® (Invitrogen®) and CellROX® deep red (Invitrogen®). In some embodiments, the detecting agent used herein is activated to produce a detectable signal by UV light, such as having a wavelength of from 100 nm to 400 nm, or of from 200 nm to 400 nm, or of from 300 to 400 nm. Examples of suitable detecting agents activated by UV light include luminol, which has an excitation wavelength of 355 nm and an emission wavelength of 411 nm. In some embodiments, the detecting agent used herein is not activated to produce a detectable signal by UV light. In one embodiment, the detecting agent is a naturally occurring antioxidant compound.

In some embodiments, the detecting agent is a positive detecting agent, i.e., the agent gives a positive signal such as fluorescence when ROSCC is present. Examples of such detecting agents include MitoSOX® and CellROX® deep red (marketed by Invitrogen), which both fluoresce in the presence of certain reactive oxidant species on excitation with visible light. Such detecting agents may be referred to as “activated” detecting agents, as they are activated to fluoresce in the presence of ROSCC. In other embodiments, the detecting agent is a negative detecting agent, i.e., its signal remains detectable until extinguished in the presence of ROSCC. Examples of such detecting agents include nitroxide-dye conjugates (see Morrow et al. Free Radic Biol Med. 2010 Jul. 1; 49(1):67-76) and beta-carotene. Such detecting agents may be referred to as “deactivated” detecting agents, as their fluorescence is deactivated in the presence of ROSCC. In some embodiments, the negative detecting agent is a naturally occurring antioxidant present in sperm. In such embodiments, the negative detecting agent ceases to fluoresce after oxidation, such as in the presence of ROSCC. It will be understood that the negative detecting agent must be excited by light, such as by visible or UV light, to observe the fluorescence and therefore loss of fluorescence upon oxidation, such as in the presence of ROSCC.

As sperm cells with levels of reactive oxidant species falling outside a biologically ideal range are infertile, and very high levels of reactive oxidant species are associated with sperm cell death, in certain embodiments, the methods herein employ a detecting agent that neutralises reactive oxidant species—in other words, a detecting agent that chemically removes the reactive, radical and/or oxidant properties of a reactive oxidant species. By way of explanation, a detecting agent may be chosen whose detectable signal, in one embodiment fluorescence, is activated on neutralisation of reactive oxidant species. Examples of such detecting agents include MitoSOX® and CellROX® deep red. In such embodiments, the methods herein may advantageously in some embodiments reduce the concentration of reactive oxidant species in the sorted sperm, thereby improving sperm health.

In one embodiment, the detecting agent comprises a fluorescing dye. In some embodiments, the fluorescence is activated by reaction with, such as neutralisation of, one or more ROSCC in the cells. In other embodiments, the fluorescence is quenched by reaction with, such as neutralisation of, one or more ROSCC in the cells. Examples of fluorescing dyes suitable for use as detecting agents herein include MitoSOX® and CellROX® deep red.

In one embodiment, the detecting agent produces a detectable signal sufficient to identify sperm as, or sort sperm into, X-CBS or Y-CBS cells after incubation with sperm for a period of less than 90 min, such as less than 75 min, less than 60 min, less than 45 min, less than 30 min, or less than 15 min, or for a period of from 5 to 90 min, or from 10 to 75 min, or from 10 to 30 min, or from 15 to 30 min, or from 30 to 60 min.

In one embodiment, the detecting agent is selective for a target ROSCC. In some embodiments, the target ROSCC is predetermined for sperm of a certain species, or sperm sampled from a particular part of a male reproductive tract, as being a ROSCC for which identifying or sorting of the sperm cells is optimised, such as because the determined difference in levels of the target ROSCC between X-CBS and Y-CBS cells is greatest and/or overlap between ROSCC levels in X-CBS and Y-CBS cell subpopulations is minimised. An example of such a detecting agent includes MitoSOX®, which is highly selective for mitochondrial superoxide.

The step of identifying or sorting in the methods described herein is according to the level of ROSCC in the sperm cells. In one embodiment, the X-CBS cells have determined levels of ROSCC that are detectably different to the levels of ROSCC in Y-CBS cells, and thus the step of identifying or sorting in the methods described herein may comprise identifying or sorting into X-CBS cells having detectably different determined levels of ROSCC compared to Y-CBS cells. In one embodiment, the determined level of ROSCC in each X-CBS cell differs by at least 5%, at least 10%, at least 20%, at least 40%, at least 50%, at least 60%, at least 75%, at least 100%, at least 200%, or at least 300% or more, or by from 5% to 500%, or by from 100% to 300%, or by from 25% to 100%, or by from 250 to 400%, from the determined level of ROSCC in each Y-CBS cell. In another embodiment, an average level of ROSCC determined across all cells in the X-CBS subpopulation differs by at least 5%, at least 10%, at least 20%, at least 40%, at least 50%, at least 60%, at least 75%, at least 100%, at least 200%, or at least 300% or more, or by from 5% to 500%, or by from 100% to 300%, or by from 25% to 100%, or by from 250 to 400%, from an average level of ROSCC determined across all cells in the Y-CBS subpopulation.

In one embodiment, the detectably different determined level is a detectably higher level of ROSCC in X-CBS cells compared to Y-CBS cells. In embodiments where X-CBS cells have higher levels of ROSCC than Y-CBS cells, the methods herein may comprise identifying at least a portion of the plurality of sperm cells as, or sorting at least a portion of the plurality of sperm cells into, X-CBS cells each having a higher determined level of ROSCC than the level of ROSCC in each Y-CBS cell. In one embodiment, each X-CBS cell has an at least 5% higher, at least 10% higher, at least 20% higher, at least 40% higher, at least 50% higher, at least 75% higher, at least 100% higher, at least 200% higher, or at least 300% higher, or at least 400% higher, or at least 500% higher, or at least 600% higher, or at least 700% higher, or at least 800% or more higher determined level of ROSCC than each Y-CBS cell, or a from 5% to 800% higher, or from 100% to 300%, or from 25% to 100% higher, or from 250 to 400% higher, or from 100 to 800% higher, or from 500 to 800% higher determined level of ROSCC than each Y-CBS cell. In some embodiments, “% higher” is measured as:

[(ROSCC level_{X-CBS}−ROSCC level_{Y-CBS})/ROSCC level_{Y-CBS}]×100.

In another embodiment, an average level of ROSCC determined across all cells in the X-CBS is least 5%, at least 10% higher, at least 20% higher, at least 40% higher, at least 50% higher, at least 75% higher, at least 100% higher, at least 200% higher, or at least 300% higher, or at least 400% higher, or at least 500% higher, or at least 600% higher, or at least 700% higher, or at least 800% or more higher than an average level of ROSCC determined across all cells in the Y-CBS, or is from 5% to 500% higher, or from 100% to 300%, or from 25% to 100% higher, or from 250 to 400% higher, or from 100 to 800% higher, or from 500 to 800% higher than an average level of ROSCC determined across all cells in the Y-CBS.

As used herein, the term “identifying” refers to a process through which certain cells are distinguished from others contained in a sample. As used herein, the term “sorting” refers to a process through which certain cells are identified or selected and then separated from others contained in a sample. In the present disclosure, the basis for identification or selection is the determined level of ROSCC. In one embodiment, the methods herein comprise identifying a portion of the plurality of cells as, or sorting a portion of the plurality of cells into, X-CBS cells, such as an X-CBS cell subpopulation. In such embodiments, cells in the plurality not identified as, or sorted into, the X-CBS cell subpopulation may be subjected to further identification, sorting or discarded. In another embodiment, the methods herein comprise identifying a portion of the plurality of cells as, or sorting a portion of the plurality of cells into, Y-CBS cells, such as a Y-CBS subpopulation. In such embodiments, cells in the plurality not identified as, or sorted into, the Y-CBS cell subpopulation may be subjected to further identification, sorting or discarded. In one embodiment, a portion of the plurality of cells is identified as, or sorted into, X-CBS cells and a separate portion of the same plurality of cells is identified as, or sorted into, Y-CBS cells, such as into a separate X-CBS subpopulation and a separate Y-CBS subpopulation. In such embodiments, cells in the plurality not identified as, or sorted into, the X-CBS cell subpopulation or the Y-CBS cell subpopulation may be subjected to further identification, sorting or discarded. In one embodiment, all or substantially all cells in the plurality are identified as, or sorted into, X-CBS or Y-CBS cells, such as up to 99%, or up to 98%, or up to 95%, of cells are sorted into X-CBS or Y-CBS.

In one embodiment, the level of ROSCC is determined in the methods herein using a flow cytometer. In one embodiment, the level of ROSCC is determined in a microfluidic device. In one embodiment, the level of ROSCC is determined based on a physiological response of the sperm cells, such as sperm motility. In one embodiment, the level of ROSCC is determined in situ in a flow cytometer or microfluidic device prior to sorting, such as immediately prior to sorting, or in order to activate sorting. In one embodiment, the sorting is performed in a flow cytometric sorter. In such embodiments, the X-CBS and Y-CBS cells may be sorted optionally by gating, such as by gating an X-CBS cell subpopulation and a Y-CBS cell subpopulation each having different levels of ROSCC. In one embodiment, the sperm are sorted based on an average level of ROSCC of a group of cells, such as an average level of ROSCC for a cell subpopulation. In another embodiment, the sorting is performed in a microfluidic sorting device. In another embodiment, enriching, or isolating at least a portion of identified X-CBS or Y-CBS cells from the starting sperm sample to produce an enriched sperm sample, is performed using a microfluidic sorting device.

Persons of skill in the art will be familiar with the operation and function of a flow cytometer and methods by which one may be employed in the methods of the present disclosure. In brief, a flow cytometer has tubes with a current of aqueous solution running through it in which cells are suspended. The cells flow through the aqueous stream until they come to a laser and a detector, at which time the instrument delivers either a slow continuous stream or a drop of solution to present to the detector containing isolated cells, whose scattering or fluorescence signal can be measured. The signal may then be used to activate a sorting mechanism, such as application of charge or a magnet, to separate cells into different collection receptacles.

Persons of skill in the art will be familiar with the operation and function of microfluidic devices and methods by which one may be employed in the methods of the present disclosure. In brief, microfluidic devices utilise a pattern of microchannels that are moulded or engraved into a chip. The microchannels are linked to larger cavities through which fluids may be injected into and/or evacuated from the chip. By adjustment of microchannel pattern and/or flow characteristics to or from the cavities, cells can be directed into particular paths for sorting and collection or disposal. Examples of suitable microfluidics devices for use in the methods described herein may include Wolf® and Wolf G2® Cell Sorters, manufactured by NanoCellect Biological.

The methods described herein may further comprise providing identified or sorted X-CBS cells and/or Y-CBS cells separately in an enriched or purified form. Naturally occurring sperm samples prior to sorting generally comprise, but are not limited to, approximately 50% X-CBS and 50% Y-CBS cells. Other variations in the sperm cell X-CBS:Y-CBS ratio prior to sorting according to the methods herein are described above.

In one embodiment, an enriched sperm population produced according to certain methods described herein comprises a relatively greater proportion of either X-CBS or Y-CBS cells in the sorted sperm relative to the sperm population pre-sorting (also referred to herein as a starting sperm sample or simply as a sperm sample). In other embodiments, an enriched sperm population produced according to certain methods described herein may comprise, by number, 60% or greater, 70% or greater, 80% or greater, 90% or greater, 95% or greater, or 99% or greater X-CBS or Y-CBS cells as a percentage of the total cell content of a sample. In one embodiment, a purified sperm population comprises either only X-CBS cells or only Y-CBS cells.

In one embodiment, the methods described herein further comprise providing X-CBS and/or Y-CBS cells, either after identification or sorting, separately in an enriched or purified form having a minimum purity of at least 90% X-CBS or at least 90% Y-CBS cells, such as at least 95% X-CBS or at least 95% Y-CBS cells, or at least 99% X-CBS or at least 95% Y-CBS cells. In some embodiments, these levels of enrichment or purity are achieved using a detecting agent activated by visible light, and/or by a detecting agent that neutralises reactive oxidant species, and/or under conditions where the detecting agent is incubated with sperm for a period of less than 90 min, such as less than 75 min, such as less than 60 min, less than 45 min, less than 30 min, or less than 15 min, or for a period of from 5 to 90 min, or from 10 to 75 min, or from 10 to 30 min, or from 15 to 30 min, or from 30 to 60 min. In one embodiment, the methods herein further comprise providing the X-CBS and/or Y-CBS cells after identification or sorting separately in a cryopreserved form. Methods of cryopreserving sperm will be known to those of skill in the art.

In one embodiment, the method of sorting sperm cells described herein is a method of sex sorting sperm cells. In another embodiment, the method of sorting sperm cells described herein is a sperm sorting method. In one embodiment, the method of sorting sperm cells described herein is a method of separating sperm cells into X-CBS and/or Y-CBS cells. In another embodiment, the method of sorting sperm cells described herein is a sperm sorting method for separating sperm cells into X-CBS and/or Y-CBS cells. In one embodiment, the method of sorting sperm cells described herein is a method of enriching X-CBS or Y-CBS cell concentration in a sperm cell sample. In another embodiment, the method of sorting sperm cells described herein is a method of purifying X-CBS or Y-CBS cells in a sperm cell sample.

As will be evident from the foregoing discussion, the present disclosure also relates to an enriched or purified population of X-CBS cells produced by identifying or sorting sperm cells according to the methods described herein. In some embodiments, the enriched or purified population has a minimum purity of at least 75%, such as at least 80%, at least 85%, at least 90%, at least 95% or at least 99% X-CBS cells as a percentage of the total cell population of the enriched sample. The present disclosure also relates to an enriched or purified population of Y-CBS cells produced by identifying or sorting sperm cells according to the methods described herein. In some embodiments, the enriched or purified population has a minimum purity of at least 75%, such as at least 80%, at least 85%, at least 90%, at least 95% or at least 99% Y-CBS cells as a percentage of the total cell population of the enriched sample.

In another aspect, described herein is a method of identifying X-chromosome bearing sperm (X-CBS) or Y-chromosome bearing sperm (Y-CBS) cells in a sperm sample, comprising: determining a level of a Reactive Oxidant Species and/or a Cellular Change mediated thereby (ROSCC) in each of a plurality of sperm cells in a starting sperm sample; based on the determined ROSCC levels, identifying at least a portion of the plurality of sperm cells in the starting sperm sample as X-CBS or Y-CBS cells.

In one embodiment, the starting sperm sample comprises a mixture of X-CBS and Y-CBS cells, such as a mixture as described above for the “plurality of sperm”. Other terms such as “determining”, “level”, “ROSCC”, “at least a portion” are as described above. The method may comprise isolating at least a portion of the identified X-CBS or Y-CBS cells from the starting sperm sample to produce an enriched sperm sample comprising the at least portion of isolated, identified X-CBS or Y-CBS cells. The enriched sperm sample may comprise at least 90% X-CBS cells, or at least 99% X-CBS cells, or may comprise at least 90% Y-CBS cells, or at least 99% Y-CBS cells. Isolating may comprise using a flow cytometric sorter, optionally with gating, or using a microfluidic sorting device. The method may alternatively further comprise inducing selective motility, infertility or non-viability in either the identified X-CBS or Y-CBS cells in the starting sperm sample to produce a motility, fertility or viability-modified sperm sample. Inducing selective motility or infertility may take the form of inducing non-motility or inducing an inability to undergo capacitation in either the identified X-CBS or Y-CBS cells, effectively rendering that portion of the sperm sample unable to produce offspring without the need for physical separation/removal of the unwanted CBS cells. Inducing selective infertility in the form of non-viability may take the form of inducing cell death in the identified X-CBS or Y-CBS cells. In one embodiment, inducing selective infertility or non-viability exploits a difference in determined levels of one or more ROSCC between the identified X-CBS and Y-CBS cells.

The present disclosure also provides for use of X-CBS or Y-CBS cells identified or sorted according to the methods described herein, or for use of samples enriched in X-CBS or Y-CBS cells produced according to the methods herein, in an assisted reproductive technology. An assisted reproductive technology may include any laboratory or clinical technology applied to isolated gametes (oocytes or sperm) for the purposes of reproduction. Such technologies include in vitro fertilization (IVF; aspiration of an oocyte, fertilization in the laboratory and transfer of the embryo into a recipient), artificial insemination (AI: placement of sperm into the pelvic cavity—such as the cervix or uterine cavity); gamete intrafallopian transfer (GIFT; placement of oocytes and sperm into the fallopian tube), zygote intrafallopian transfer (ZIFT; placement of fertilized oocytes into the fallopian tube), tubal embryo transfer (TET; the placement of cleaving embryos into the fallopian tube), peritoneal oocyte and sperm transfer (POST; the placement of oocytes and sperm into the pelvic cavity), intracytoplasmic sperm injection (ICSI), testicular sperm extraction (TESE), and microsurgical epididymal sperm aspiration (MESA); or any other in vitro technique for producing embryos in humans and/or animals, such as nuclear transfer, parthenogenic activation, embryonic stem cell production, and the use of totipotent cells. In one embodiment, the assisted reproductive technology is in vitro fertilisation. In another embodiment, the assisted reproductive technology is artificial insemination. In some embodiments, sperm provided for use in an assisted reproductive technology may have a minimum enrichment or purity, such as may be at least 90% X-CBS (or Y-CBS), at least 95% X-CBS (or Y-CBS), or at least 99% X-CBS (or Y-CBS). Persons of skill in the art will be familiar with preparation required to provide sperm sorted according to the methods described herein in a form suitable for use in an assisted reproductive technology.

In one embodiment, the present disclosure relates to a non-human mammalian subject produced from the sperm identified, sorted or enriched according to the methods described herein, such as from sorted or enriched populations of X-CBS or Y-CBS cells.

Also disclosed herein is a sperm cell sorting system, comprising: a Reactive Oxidant Species and/or a Cellular Change (ROSCC) detecting agent, wherein the Cellular Change is mediated by a Reactive Oxidant Species; and, instructions to use the ROSCC detecting agent to determine a level of ROSCC in each of a plurality of sperm cells, and based on the determined ROSCC levels, sort at least a portion of the plurality of sperm cells into X-chromosome bearing sperm (X-CBS) and/or Y-chromosome bearing sperm (Y-CBS) cells. In one embodiment, the system further comprises a sperm cell incubation solution for incubating sperm cells with the ROSCC detecting agent. The sperm cell sorting system may be in the form of a kit, such as a box, container, bag, etc., with the ROSCC detecting agent and optionally sperm cell incubation solution being supplied in separate, sealed containers. In some embodiments, the system further comprises one or more components required to sort the sperm after incubation with the ROSCC detecting agent. In one embodiment, the system comprises a microfluidic sorting device for sorting the sperm that is adapted for use with the detecting agent included in the system. In one embodiment, the microfluidic device sorting is reusable. The detecting agent and ROSCC in the system are as described in the foregoing description.

In one embodiment, the system comprises two or more, such as two, three, four, five, ten, or twenty separately packaged detecting agents. In one embodiment, the system comprises two or more, such as two, three, four, five, ten, or twenty separately packaged incubating solutions. In one embodiment, the detecting agents and incubating solutions are identical. In one embodiment, the detecting agents and/or incubating solutions are included in the system in a ready to use form. In another embodiment, the detecting agents and/or incubating solutions are provided in the system in a form requiring reconstitution or dilution prior to use. Systems comprising two or more detecting agents and optionally two or more incubating solutions may be advantageously packaged with a reusable microfluidic device adapted for use with the detecting agent and incubation solution included in the system, such that a single system may analyse two or more different sperm samples at different times. In one embodiment, the detecting agents and incubating solutions are different and complementary. In such embodiments, two or more different microfluidic sorting devices may be included in the system. Other components of the kit may include storage solution(s) for the sorted or enriched sperm, as well as apparatus to assist with the administration of sorted or enriched sperm in assisted reproductive technologies, etc.

It should be understood that any numerical range recited herein is intended to include all sub-ranges subsumed therein. For example, a range of “from x to y” or “between x and y” is intended to include all sub-ranges between x and y and also range end points x and y.

As used herein, the singular forms “a,” “an,” and “the” may refer to plural articles unless specifically stated otherwise.

### EMBODIMENTS

The following embodiments form part of the present disclosure:

Embodiment 1. A method of sorting sperm cells, comprising:
determining a level of a Reactive Oxidant Species and/or a Cellular Change mediated thereby (ROSCC) in each of a plurality of sperm cells; and
based on the determined ROSCC levels, sorting at least a portion of the plurality of sperm cells into X chromosome bearing sperm (X-CBS) and/or Y chromosome bearing sperm (Y-CBS) cells.

Embodiment 2. The method of Embodiment 1, comprising sorting at least a portion of the plurality of sperm cells into X-CBS and Y-CBS cells.

Embodiment 3. The method of Embodiment 1 or Embodiment 2, wherein the ROSCC is selected from:
a reactive oxidant species selected from: a reactive oxygen species (ROS), a reactive nitrogen species (RNS), and a reactive sulfur species (RSS), or a combination of two or more thereof; and/or
a cellular change mediated by a reactive oxidant species selected from: a ROS-induced oxidative change, an RNS-induced oxidative change, and an RSS-induced oxidative change, or a combination of two or more thereof.

Embodiment 4. The method of any one of Embodiments 1 to 3, wherein determining the level of ROSCC comprises treating the plurality of sperm cells with a ROSCC detecting agent.

Embodiment 5. The method of any one of Embodiments 1 to 4, wherein determining the level of ROSCC comprises treating the plurality of sperm cells with a ROSCC detecting agent comprising a dye.

Embodiment 6. The method of any one of Embodiments 1 to 5, wherein determining the level of ROSCC comprises treating the plurality of sperm cells with a ROSCC detecting agent comprising a fluorescing dye, optionally where fluorescence is activated by reaction with one or more ROSCC in the cells.

Embodiment 7. The method of any one of Embodiments 1 to 6, wherein the ROSCC is detectable in a component of the sperm cells selected from: a lipid component, such as the cell membrane or mitochondrial membrane; a protein component, such as chromatin or an enzyme; an organelle, such as the mitochondria, an extracellular component, such as a cell surface enzyme; the cytoplasm; and DNA, or a combination of any two or more of these components.

Embodiment 8. The method of any one of Embodiments 1 to 7, wherein determining the level of ROSCC comprises treating the plurality of sperm cells with a ROSCC detecting agent selective for a target ROSCC.

Embodiment 9. The method of any one of Embodiments 1 to 8, wherein the ROSCC is a reactive oxidant species, and the reactive oxidant species is a ROS selected from superoxide, hydroxyl radical, and hydrogen peroxide.

Embodiment 10. The method of any one of Embodiments 1 to 9, wherein determining the level of ROSCC comprises treating the plurality of sperm cells with a detecting agent that detects a ROS, an RNS and/or an RSS in at least the cytoplasm of the sperm cells.

Embodiment 11. The method of any one of Embodiments 1 to 8, wherein the ROSCC is a cellular change mediated by a reactive oxidant species, wherein the cellular change is lipid peroxidation.

Embodiment 12. The method of any one of Embodiments 1 to 8 or 11, wherein determining the level of ROSCC comprises treating the plurality of sperm cells with a ROSCC detecting agent that detects a cellular change mediated by a reactive oxidant species, wherein the cellular change is in a lipid component of the sperm cells.

Embodiment 13. The method of any one of Embodiments 1 to 12, wherein the level of ROSCC determined in each X-CBS cell differs by at least 5%, at least 20%, at least 40%, or at least 60% from the level of ROSCC determined in each Y-CBS cell.

Embodiment 14. The method of any one of Embodiments 1 to 13, wherein the level of ROSCC determined in each X-CBS cell is at least 5%, at least 20%, at least 40%, or at least 60% higher than the level of ROSCC determined in each Y-CBS cell.

Embodiment 15. The method of any one of Embodiments 1 to 12, wherein an average level of ROSCC determined across all cells in the X-CBS differs by at least 5%, at least 20%, at least 40%, or at least 60% from an average level of ROSCC determined across all cells in the Y-CBS.

Embodiment 16. The method of any one of Embodiments 1 to 12 or 15, wherein an average level of ROSCC determined across all cells in the X-CBS is least 5%, at least 20%, at least 40%, or at least 60% higher than an average level of ROSCC determined across all cells in the Y-CBS.

Embodiment 17. The method of any one of Embodiments 1 to 16, wherein the plurality of sperm cells are obtained from the proximal cauda epididymis, the distal cauda epididymis, the vas deferens, or ejaculated semen of a subject.

Embodiment 18. The method of any one of Embodiments 1 to 17, wherein the plurality of sperm cells are obtained from a mammalian subject.

Embodiment 19. The method of any one of Embodiments 1 to 18, wherein the plurality of sperm cells are obtained from a non-human mammalian subject.

Embodiment 20. The method of any one of Embodiments 1 to 19, wherein the sorting comprises using a flow cytometric sorter or a microfluidic sorting device.

Embodiment 21. The method of any one of Embodiments 1 to 20, wherein the sorting comprises using a flow cytometric sorter and the X-CBS and/or Y-CBS are sorted by gating.

Embodiment 22. The method of any one of Embodiments 1 to 21, wherein the determining is performed in situ in a flow cytometer or microfluidic device prior to sorting.

Embodiment 23. The method of any one of Embodiments 1 to 22, further comprising:
providing the sorted X-CBS and/or Y-CBS separately in an enriched or purified form.

Embodiment 24. The method of any one of Embodiments 1 to 23, further comprising:
providing the sorted X-CBS and/or Y-CBS cells separately in an enriched or purified form having a minimum purity of at least 90% X-CBS or at least 90% Y-CBS cells.

Embodiment 25. The method of any one of Embodiments 1 to 24, further comprising:
providing the sorted X-CBS and/or Y-CBS cells separately in a cryopreserved form.

Embodiment 26. The method of any one of Embodiments 1 to 25, wherein the plurality of sperm cells comprise a mixture of X-CBS and Y-CBS cells.

Embodiment 27. An enriched or purified population of X chromosome bearing sperm (X-CBS) cells sorted according to the method of any one of Embodiments 1 to 26.

Embodiment 28. The enriched or purified population of X-CBS cells according to Embodiment 27 having a minimum purity of at least 90% X-CBS cells.

Embodiment 29. An enriched or purified population of Y chromosome bearing sperm (Y-CBS) cells sorted according to the method of any one of Embodiments 1 to 26.

Embodiment 30. The enriched or purified population of Y-CBS cells according to Embodiment 29 having a purity of at least 90% Y-CBS cells.

Embodiment 31. A method of identifying X chromosome bearing sperm (X-CBS) or Y chromosome bearing sperm (Y-CBS) cells in a sperm sample, the method comprising: determining a level of a Reactive Oxidant Species and/or a Cellular Change mediated thereby (ROSCC) in each of a plurality of sperm cells in a starting sperm sample; and based on the determined ROSCC levels, identifying at least a portion of the plurality of sperm cells in the starting sperm sample as X-CBS or Y-CBS cells.

Embodiment 32. The method of Embodiment 31, wherein the starting sperm sample comprises a mixture of X-CBS and Y-CBS cells.

Embodiment 33. The method of Embodiment 31 or 32, wherein the ROSCC is selected from: a reactive oxidant species selected from: a reactive oxygen species (ROS), a reactive nitrogen species (RNS), and a reactive sulfur species (RSS), or a combination of two or more thereof; and/or a cellular change mediated by a reactive oxidant species selected from: a ROS-induced oxidative change, an RNS-induced oxidative change, and an RSS-induced oxidative change, or a combination of two or more thereof.

Embodiment 34. The method of any one of Embodiments 31 to 33, wherein determining the level of ROSCC comprises treating the plurality of sperm cells with a ROSCC detecting agent.

Embodiment 35. The method of any one of Embodiments 31 to 34, wherein determining the level of ROSCC comprises treating the plurality of sperm cells with a ROSCC detecting agent comprising a dye.

Embodiment 36. The method of any one of Embodiments 31 to 35, wherein determining the level of ROSCC comprises treating the plurality of sperm cells with a ROSCC detecting agent comprising a fluorescing dye, optionally where fluorescence is activated by reaction with one or more ROSCC in the cells.

Embodiment 37. The method of any one of Embodiments 31 to 36, wherein the ROSCC is detectable in a component of the sperm cells selected from: a lipid component, such as the cell membrane or mitochondrial membrane; a protein component, such as chromatin or an enzyme; an organelle, such as the mitochondria, an extracellular component, such as a cell surface enzyme; the cytoplasm; and DNA, or a combination of any two or more of these components.

Embodiment 38. The method of any one of Embodiments 31 to 37, wherein determining the level of ROSCC comprises treating the plurality of sperm cells with a ROSCC detecting agent selective for a target ROSCC.

Embodiment 39. The method of any one of Embodiments 31 to 38, wherein the ROSCC is a reactive oxidant species, and the reactive oxidant species is a ROS selected from superoxide, hydroxyl radical, and hydrogen peroxide.

Embodiment 40. The method of any one of Embodiments 31 to 39, wherein determining the level of ROSCC comprises treating the plurality of sperm cells with a detecting agent that detects a ROS, an RNS and/or an RSS in at least the cytoplasm of the sperm cells.

Embodiment 41. The method of any one of Embodiments 31 to 39, wherein the ROSCC is a cellular change mediated by a reactive oxidant species, wherein the cellular change is lipid peroxidation.

Embodiment 42. The method of any one of Embodiments 31 to 39 or 41, wherein determining the level of ROSCC comprises treating the plurality of sperm cells with a ROSCC detecting agent that detects a cellular change mediated by a reactive oxidant species, wherein the cellular change is in a lipid component of the sperm cells.

Embodiment 43. The method of any one of Embodiments 31 to 42, wherein the determined level of ROSCC in each cell identified as X-CBS differs from the determined ROSCC levels of each cell identified as Y-CBS by at least 5%, at least 20%, at least 40%, or at least 60%.

Embodiment 44. The method of any one of Embodiments 31 to 43, wherein the determined level of ROSCC in each cell identified as X-CBS is at least 5%, at least 20%, at least 40%, or at least 60% higher than the level of ROSCC determined in each cell identified as Y-CBS.

Embodiment 45. The method of any one of Embodiments 31 to 43, wherein an average level of ROSCC determined across the portion of cells identified as X-CBS differs from an average level of ROSCC determined across the portion of cells identified as Y-CBS by at least 5%, at least 20%, at least 40%, or at least 60%.

Embodiment 46. The method of any one of Embodiments 31 to 43 or 45, wherein an average level of ROSCC determined across the portion of cells identified as X-CBS is least 5%, at least 20%, at least 40%, or at least 60% higher than an average level of ROSCC determined across the portion of cells identified as Y-CBS.

Embodiment 47. The method of any one of Embodiments 31 to 46, wherein the sperm cells are obtained from the proximal cauda epididymis, the distal cauda epididymis, the vas deferens, or ejaculated semen of a subject.

Embodiment 48. The method of any one of Embodiments 31 to 47, wherein the sperm cells are obtained from a mammalian subject.

Embodiment 49. The method of any one of Embodiments 31 to 48, wherein the sperm cells are obtained from a non-human mammalian subject.

Embodiment 50. The method of any one of Embodiments 31 to 49, further comprising isolating at least a portion of the identified X-CBS or Y-CBS cells from the starting sperm sample to produce an enriched sperm sample comprising the at least portion of isolated, identified X-CBS or Y-CBS cells.

Embodiment 51. The method of Embodiment 50, wherein the enriched sperm sample comprises at least 90% X-CBS cells, optionally at least 99% X-CBS cells, or comprises at least 90% Y-CBS cells, optionally at least 99% Y-CBS cells.

Embodiment 52. The method of Embodiment 50 or Embodiment 51, wherein isolating comprises using a flow cytometric sorter or a microfluidic sorting device.

Embodiment 53. The method of any one of Embodiments 50 to 52, wherein isolating comprises using a flow cytometric sorter and gating either the identified X-CBS or Y-CBS cells.

Embodiment 54. The method of any one of Embodiments 50 to 53, wherein the determining is performed in situ in a flow cytometer or microfluidic sorting device prior to isolating the at least a portion of identified X-CBS or Y-CBS cells.

Embodiment 55. The method of any one of Embodiments 31 to 49, further comprising inducing selective motility, infertility or non-viability in either the identified X-CBS or Y-CBS cells in the starting sperm sample to produce a motility, fertility or viability-modified sperm sample.

Embodiment 56. The method of any one of Embodiments 31 to 55, further comprising:
cryopreserving the enriched sperm sample.

Embodiment 57. Use of sperm sorted according to the method of any one of Embodiments 1 to 26, or an enriched or purified population of X chromosome bearing sperm (X-CBS) or Y chromosome bearing sperm (Y-CBS) cells according to any one of Embodiments 27 to 30, or sperm identified as X-CBS or Y-CBS according to the method of any one of Embodiments 31 to 56, in an assisted reproductive technology.

Embodiment 58. A non-human mammalian subject produced from sperm sorted according to the method of any one of Embodiments 1 to 26, an enriched or purified population of X chromosome bearing sperm (X-CBS) or Y chromosome bearing sperm (Y-CBS) cells according to any one of Embodiments 27 to 30, or sperm identified as X-CBS or Y-CBS according to the method of any one of Embodiments 31 to 56.

Embodiment 59. A sperm cell sorting system, comprising: a Reactive Oxidant Species and/or a Cellular Change (ROSCC) detecting agent, wherein the Cellular Change is mediated by a Reactive Oxidant Species; and instructions to use the ROSCC detecting agent to determine a level of ROSCC in each of a plurality of sperm cells, and based on the determined ROSCC levels, sorting at least a portion of the plurality of sperm cells into X chromosome bearing sperm (X-CBS) and/or Y chromosome bearing sperm (Y-CBS) cells.

Embodiment 60. The sperm cell sorting system of Embodiment 59, further comprising a sperm cell incubation solution for incubating sperm cells with the ROSCC detecting agent.

### EXAMPLES

### Example 1

### 1.1. Methods and Materials

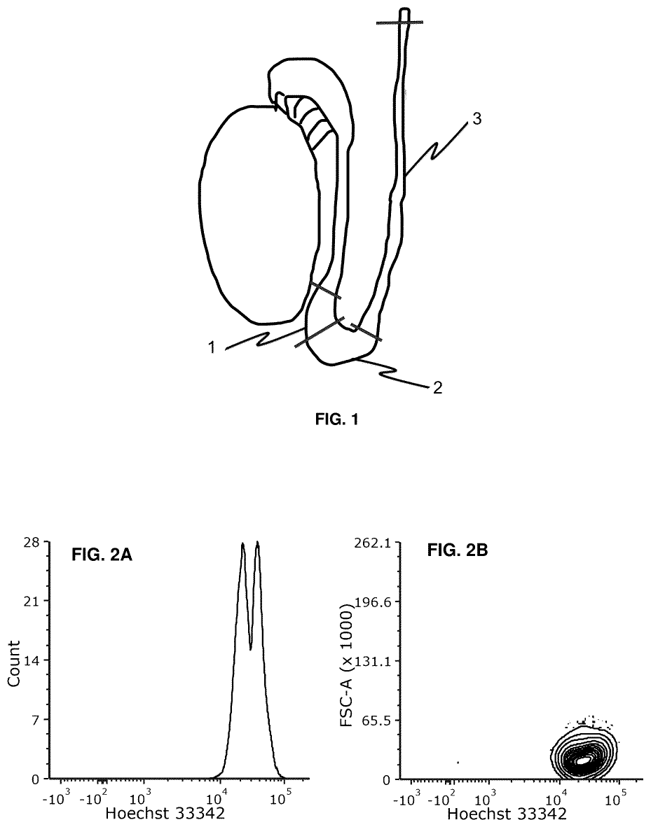

C57BL/6 strain mice were obtained and humanely killed via cervical dislocation prior to removal of the epididymis and vas deferens. The cauda was sectioned into two main regions: the ‘proximal cauda’ is the region of the cauda epididymis adjacent to the corpus epididymis and the ‘distal cauda’ which was the section adjacent to the vas deferens (see FIG. 1). The vas deferens was sectioned at the cauda and as close to the ejaculatory duct as possible. The tissue from the epididymis and vas deferens was covered in prewarmed phosphate buffered saline at pH 7.4 (PBS). The tissue was gently torn open, and the semen washed from the tissue. The semen PBS mix was gently combined and centrifuged at 400 g. The supernatant was discarded; the sperm were resuspended and subjected to one of the following protocols:

1.1a. Mitochondrial Superoxide

Mitochondrial superoxide was measured by incubating sperm at 37° C. with MitoSOX® red mitochondrial superoxide indicator (excitation 488 nm emission 585 nm; ThermoFisher Scientific Australia M36008) at 1.25 μM, in the dark for 10 min. The sperm were then washed by diluting 1:5 in prewarmed PBS, prior to centrifugation at 400 g, resuspension in 200 μL of prewarmed PBS and immediate analysis via flow cytometry.

1.1 b. Cytoplasmic Reactive Oxygen Species

Cytoplasmic ROS was measured by incubating sperm at 37° C. with the broad-spectrum ROS detecting molecular probe CellROX® deep red (excitation 644 nm emission 665 nm; ThermoFisher Scientific Australia, C10491) at 2.5 μM, in the dark for 30 min. The sperm were then washed by diluting 1:5 in prewarmed PBS, prior to centrifugation at 400 g, resuspension in 200 μL of prewarmed PBS and immediate analysis via flow cytometry.

1.1c. Lipid Peroxidation

Lipid peroxidation was measured by incubating sperm at 37° C. with the lipid peroxidation sensor Bodipy 581/591 C11 (excitation 488 nm emission 585 nm; ThermoFisher Scientific Australia, D3861) at 1.25 μM, in the dark for 30 min. The sperm were then washed by diluting 1:5 in prewarmed PBS, prior to centrifugation at 400 g, resuspension in 200 μL of prewarmed PBS and immediate analysis via flow cytometry.

### 1.2. Flow Cytometry

Samples were analysed on a BD FACS Canto II. The forward scatter, side scatter and PMT voltages were optimised to place the negative controls (unstained sperm) in approximately the second decade of the log scale. Flow rate was set to low and 10,000 events were recorded. Data was analysed with FCS Express 6 plus software.

### 1.3. Results and Discussion

In all locations of the reproductive tract, X-CBS have more mitochondrial superoxide, more cytoplasmic ROS and a higher degree of lipid peroxidation than Y-CBS. In comparison to Hoechst 33342 as a dye to assist in sorting X-CBS and Y-CBS, mitochondrial superoxide is superior. The degree of lipid peroxidation and cytoplasmic ROS are comparable to the current Hoechst method when used simultaneously (see FIGS. 2-7).

With reference to FIG. 1, there is shown a diagrammatic representation of a testis showing the three locations of the reproductive tract from which sperm were obtained for experiments as described in the Examples above. The cauda epididymis was halved; the part of the cauda epididymis adjacent to the corpus epididymis 1 was termed “Proximal Cauda”, the part of the cauda adjacent to the vas deferens 2 was termed “Distal cauda” and the vas deferens from the cauda to as close to the ejaculatory duct as possible 3 was termed “vas deferens”. Also shown is the head of the epididymis 13, the seminiferous tubules 10, the tunica albuginea 11, the body of the epididymis 12, the tail of the epididymis 15 and the vas deferens 14.

With reference to FIG. 2, data shows the degree of separation between X-CBS and Y-CBS using current sperm sorting technology. Sperm were stained with Hoechst 33342 at 111 μM for 30 min. Populations of X-CBS are 1.9 times brighter in fluorescence intensity than Y-CBS. The histogram in FIG. 2a) shows moderate overlap in fluorescence intensity, whilst the contour plot in FIG. 2b) reveals that there is a large amount of overlap between the two chromosome bearing sperm types.

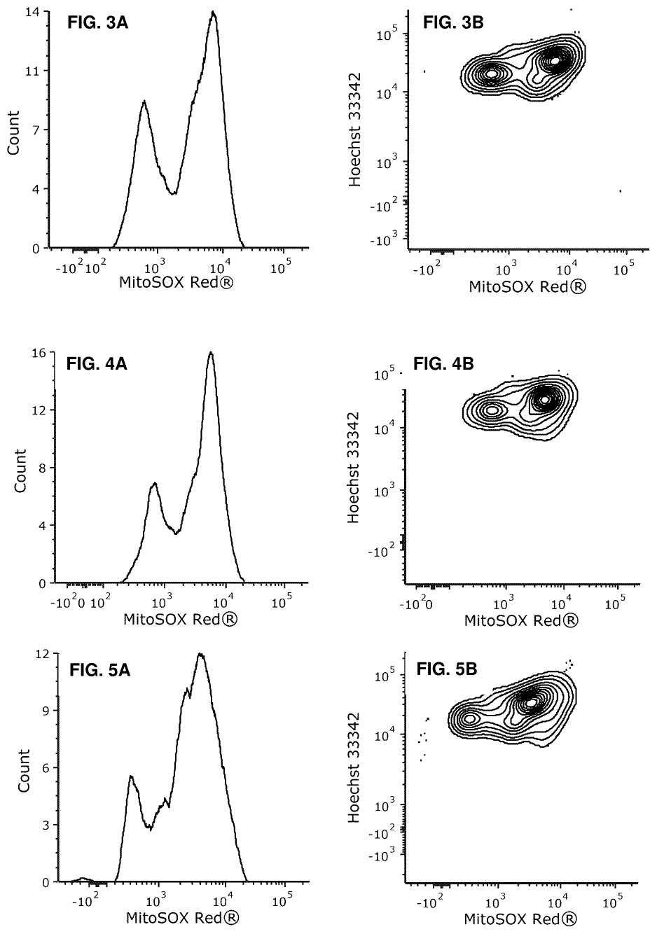

FIG. 3 shows sorting data from sperm cells isolated from the Proximal Cauda epididymis with ROSCC levels detected by MitoSOX® according to the methods described herein. These data show the superiority of superoxide as a detection characteristic for the separation of X-CBS and Y-CBS compared with available technology. The fluorescence intensity of X-CBS as shown in FIG. 3 is 8.5 times higher than Y-CBS in sperm in the proximal cauda epididymis. The histogram in FIG. 3a) demonstrates a minor degree of overlap in fluorescence intensity between X-CBS and Y-CBS. The contour plot in FIG. 3b) reveals that the order of magnitude of cells overlapping in the two peaks in 3a) is minor.

FIG. 4 shows sorting data from sperm cells isolated from the Distal Cauda epididymis with ROSCC levels detected by MitoSOX® according to the methods described herein. These data reinforce the superiority of superoxide as a detection characteristic for the separation of X-CBS and Y-CBS compared with available technology. The fluorescence intensity of X-CBS in FIG. 4 is 6.5 times higher than Y-CBS in sperm in the distal cauda epididymis. The histogram in FIG. 4a) demonstrates a minor degree of overlap in fluorescence intensity between X-CBS and Y-CBS. The contour plot in FIG. 4b) reveals that the order of magnitude of cells overlapping in the two peaks in FIG. 4a) is minor, with only a few cells.

FIG. 5 shows sorting data from sperm cells isolated from the Vas Deferens with ROSCC levels detected by MitoSOX® according to the methods described herein. These data reinforce the superiority of superoxide as a detection characteristic for the separation of X-CBS and Y-CBS compared with available technology. The fluorescence intensity of X-CBS in FIG. 5 is 7.6 times higher than Y-CBS in sperm in the Vas Deferens. The histogram in FIG. 5a) demonstrates very little overlap in fluorescence intensity between X-CBS and Y-CBS. The contour plot in FIG. 5b) reveals that the order of magnitude of cells overlapping in the two peaks in FIG. 5a) is minor, with only a few cells.

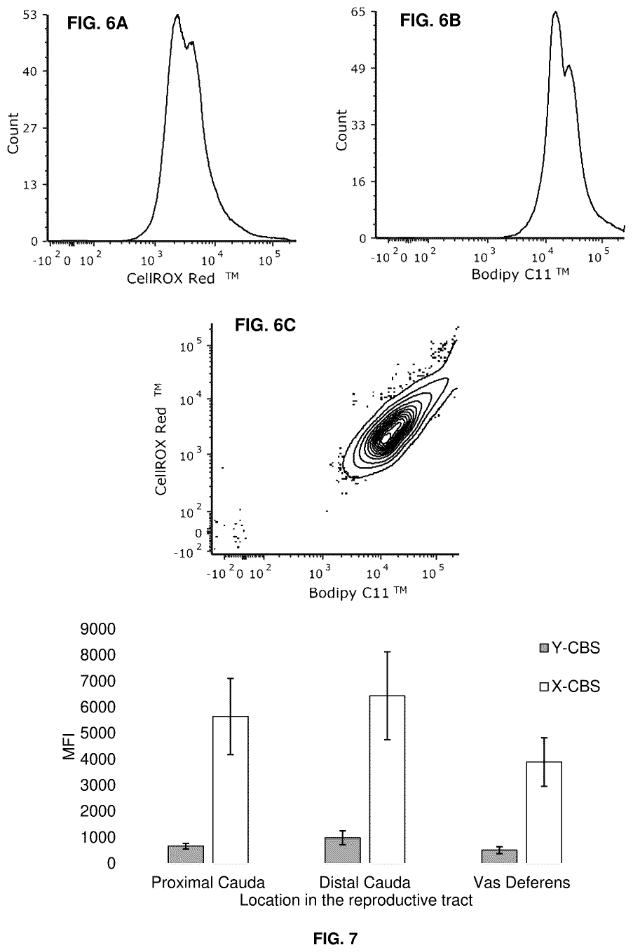

With reference to FIG. 6a), data shows low resolution of X-CBS and Y-CBS when sorted via cytoplasmic ROS detected with CellROX deep Red®, and FIG. 6b) similarly low resolution of X-CBS and Y-CBS when sorted via membrane lipid peroxidation detected with Bodipy C11®. However, in FIG. 6c) high resolution can be achieved when sperm are sorted via cytoplasmic ROS and lipid peroxidation. Combining cytoplasmic ROS and lipid peroxidation in a two-dimensional sort achieves resolution higher than that of Hoechst.

FIG. 7 visualises the large difference in MitoSOX® detected superoxide concentrations between X-CBS and Y-CBS as demonstrated by the histogram and contour plots in FIGS. 3-6. The striking differences in superoxide observed between X-CBS and Y-CBS and represented in FIG. 7 in unejaculated sperm using the methods described herein may allow for faster sorting times, higher viability of sperm post sort, and reduced likelihood of oxidative stress, which is a common inhibitor of fertility in mammals. Current technology, which utilises variants of the nucleotide binding Hoechst stain, results in X-CBS glowing 1.9 times more brightly than Y-CBS. The methods described herein surpass current technology by utilising superoxide, a potential inhibitor of sperm function, as a detection characteristic for sorting sperm.

### Example 2

### 2.1. Methods and Materials

2.1.1 Cryopreserved bull semen was thawed at 37° C. for 60 sec. the sample was diluted 1:3 in PBS, washed and incubated with 2.5 μM MitoSOX Red in PBS and incubated for 10 min in the dark. Samples were washed by diluting in 1:5 in PBS, prior to centrifugation at 600 g for 2 min. Samples were resuspended in 200 μL of PBS prior to analysis via flow cytometry.

2.1.2 Horse semen was purchased from Elderslie Horse Care and Spelling Tasmania. Semen was provided in Equiplus equine semen extender (Minitube, Australia) at a ratio of 1:1. Cells were concentrated by first diluting 1000 μL of extended semen in 4000 μL of PBS at pH 7.4, and centrifuging at 6000 g for 20 min. The pellet was washed a second time in 1000 μL of PBS. The pellet was then resuspended in 2.5 μM MitoSOX Red in PBS and incubated for 10 min in the dark. Samples were washed by diluting in 1:5 in PBS, prior to centrifugation at 600 g for 2 min. Samples were resuspended in 200 μL of PBS prior to analysis via flow cytometry.

2.1.3 Boar semen was purchased from Sabor Limited. 200 μL of extended semen was incubated with 5 μM MitoSOX Red in PBS and incubated for 10 min in the dark. Samples were washed by diluting in 1:5 in PBS. Prior to analysis via flow cytometry. Samples were washed by diluting in 1:5 in PBS, prior to centrifugation at 600 g for 2 min. Samples were resuspended in 200 μL of PBS prior to analysis via flow cytometry.

### 2.2. Flow Cytometry

Cells were analysed on a Beckman Dickinson BD FACS Canto II. The forward scatter, side scatter and PMT voltages were optimised to place the negative controls (unstained spermatozoa) in approximately the second decade of the log scale. Flow rate was set to low and 50,000 events were recorded. Data was analysed with FCS Express 6 plus software.

### 2.3. Results and Discussion

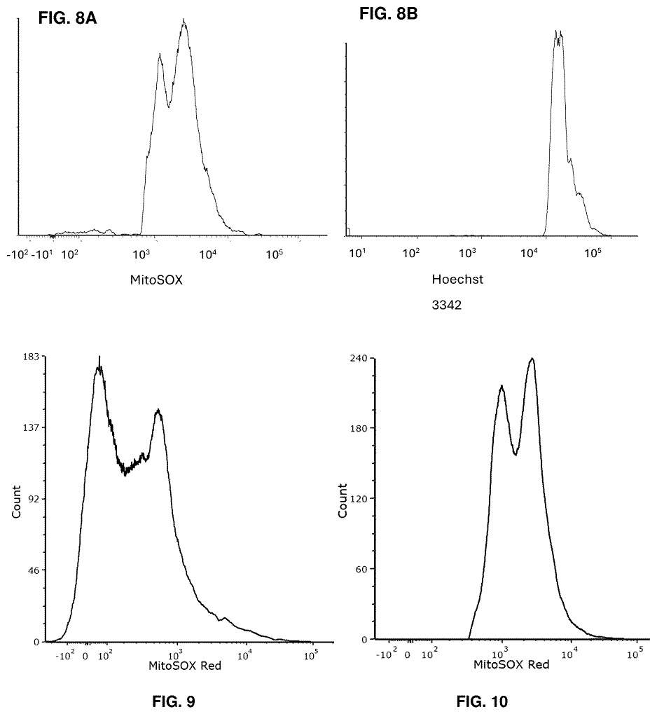

FIG. 8 shows data that visualises (a) the large difference in MitoSOX® detected superoxide concentrations between bull X-CBS and Y-CBS as demonstrated by the histogram compared to (b) sperm stained with Hoechst 33342. Cryopreserved semen is difficult to separate, and there are typically many steps involved in increasing Hoechst permeability and reducing the signal interference from dead sperm (caused by the freezing process). The methods of the present invention give superior resolution to the different X-CBS and Y-CBS subpopulations compared to current Hoechst methods.

FIG. 9 shows data that visualises the large difference in MitoSOX® detected superoxide concentrations between horse X-CBS and Y-CBS as demonstrated by the histogram.

FIG. 10 shows data that visualises the large difference in MitoSOX® detected superoxide concentrations between boar X-CBS and Y-CBS as demonstrated by the histogram.

While the present invention is described with reference to the above examples, it is to be understood that the examples are illustrative of and not limiting to the invention described herein.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

1. A method of differentiating between X-chromosome bearing sperm (X-CBS) and Y-chromosome bearing sperm (Y-CBS) cells in a sperm sample, the method comprising:
determining a level of a Reactive Oxidant Species and/or a Cellular Change mediated thereby (ROSCC) in each of a plurality of sperm cells in the sperm sample; and
based on the determined ROSCC levels, differentiating between X-CBS and Y-CBS cells in at least a portion of the plurality of sperm cells.

2. The method of claim 1, wherein the ROSCC is selected from:
a reactive oxidant species selected from: a reactive oxygen species (ROS), a reactive nitrogen species (RNS), and a reactive sulfur species (RSS), or a combination of two or more thereof; and/or
a cellular change mediated by a reactive oxidant species selected from: a ROS-induced oxidative change, an RNS-induced oxidative change, and an RSS-induced oxidative change, or a combination of two or more thereof.

3. The method of claim 1, wherein determining the level of ROSCC comprises treating the plurality of sperm cells with a ROSCC detecting agent.

4. The method of claim 1, wherein determining the level of ROSCC comprises treating the plurality of sperm cells with a ROSCC detecting agent comprising a dye.

5. The method of claim 1, wherein determining the level of ROSCC comprises treating the plurality of sperm cells with a ROSCC detecting agent comprising a fluorescing dye.

6. The method of claim 1, wherein the ROSCC is detectable in a component of the sperm cells selected from: a lipid component; a protein component; an organelle; an extracellular component; the cytoplasm; and DNA, or a combination of any two or more of these components.

7. The method of claim 1, wherein determining the level of ROSCC comprises treating the plurality of sperm cells with a ROSCC detecting agent selective for a target ROSCC.

8. The method of claim 1, wherein the ROSCC is a reactive oxidant species (ROS), and the reactive oxidant species is a ROS selected from superoxide, hydroxyl radical, and hydrogen peroxide.

9. The method of claim 1, wherein determining the level of ROSCC comprises treating the plurality of sperm cells with a detecting agent that detects a ROS, an RNS and/or an RSS in at least the cytoplasm of the sperm cells.

10. The method of claim 1, wherein the ROSCC is a cellular change mediated by a reactive oxidant species, wherein the cellular change is lipid peroxidation.

11. The method of claim 1, wherein determining the level of ROSCC comprises treating the plurality of sperm cells with a ROSCC detecting agent that detects a cellular change mediated by a reactive oxidant species, wherein the cellular change is in a lipid component of the sperm cells.

12. The method of claim 1, wherein determining the level of ROSCC comprises treating the plurality of sperm cells with a ROSCC detecting agent that detects a cellular change mediated by a reactive oxidant species, wherein the cellular change is in a protein component of the sperm cells.

13. The method of claim 1, wherein an average level of ROSCC for X-CBS cells differs from an average level of ROSCC for Y-CBS cells by at least 5%, at least 20%, at least 40%, or at least 60%.

14. The method of claim 1, wherein an average level of ROSCC for X-CBS cells is at least 5%, at least 20%, at least 40%, or at least 60% higher than an average level of ROSCC for Y-CBS cells.

15. The method of claim 1, wherein the sperm cells are obtained from the proximal cauda epididymis, the distal cauda epididymis, the vas deferens, or ejaculated semen of a subject.

16. The method of claim 1, wherein the sperm cells are obtained from a mammalian subject.

17. The method of claim 1, wherein the sperm cells are obtained from a non-human mammalian subject.

18. The method of claim 1, wherein determining the level of ROSCC comprises using a flow cytometer or a microfluidic device.

19. The method of claim 1, further comprising isolating at least a portion of the differentiated X-CBS or Y-CBS cells from the sperm sample to produce an enriched or purified sperm sample.

20. The method of claim 1, wherein the determining is performed in situ in a flow cytometer or microfluidic device.

21-22. (canceled)
